(19) United States
(12) Patent Application Publication
(10) Pub. No.: US 2022/0059238 A1
(43) Pub. Date: Feb. 24, 2022

(54) SYSTEMS AND METHODS FOR GENERATING DATA QUALITY INDICES FOR PATIENTS

(71) Applicant: GE Precision Healthcare LLC, Wauwatosa, WI (US)

(72) Inventors: Yrjö Tapio Häme, Helsinki (FI); Hanna Katriina Saarinen, Helsinki (FI); Timo Markus Taneli Petäjä, Helsinki (FI)

(21) Appl. No.: 17/001,436

(22) Filed: Aug. 24, 2020

(51) Int. Cl. G16H 50/70; G16H 10/60; G16H 50/20; G16H 50/30; G16H 10/40; G16H 40/67; G06F 16/215; A61B 5/00

(52) U.S. Cl. CPC G16H 50/70; G16H 10/60; G16H 50/20; G16H 50/30; A61B 5/742; G16H 40/67; G06F 16/215; A61B 5/7221; A61B 5/7275; G16H 10/40

(57) ABSTRACT

Systems and methods are provided for generating a data quality index. In one example, a method can include detecting an input data stream for a patient from a medical device and receiving a request to generate a patient score and the data quality index for the patient with artificial intelligence instructions and the input data stream. The method can also include determining that a feature for the input data stream is unavailable for the patient, selecting imputed values from a distribution for the unavailable feature, and calculating initial scores using the imputed values from a distribution as input for the at least one feature that is unavailable. The method can include calculating the data quality index based on the initial scores and providing the data quality index to a device, wherein the data quality index indicates a reliability of the patient score.

### FIELD

Embodiments of the subject matter disclosed herein relate to generating data quality indices for patients as a diagnostic tool.

### BACKGROUND

Acute care of patients in a hospital or other medical facility may be carried out with multiple care providers per patient and may include multiple patient monitoring devices monitoring each patient. Thus, to ensure a rapid response should a patient's condition deteriorate, near-continuous monitoring of the output from the multiple monitoring devices may be necessary. Further, coordination of patient care among all the care providers may be complicated or time-consuming, further stretching care provider resources. Additionally, the patient medical information used to assess a condition of a patient may be incomplete or unavailable.

### SUMMARY

This summary introduces concepts that are described in more detail in the detailed description. It should not be used to identify essential features of the claimed subject matter, nor to limit the scope of the claimed subject matter.

In some aspects, a method for generating a data quality index can include receiving a request to generate a patient score and an associated data quality index for a patient with a set of artificial intelligence instructions and one or more input data streams. The method can also include determining that at least one feature for the one or more input data streams is unavailable for the patient and selecting one or more imputed values from a distribution for each of one or more features for the patient population. Additionally, the method can include calculating one or more initial scores using the one or more imputed values from the distribution as input for the at least one feature that is unavailable, wherein the initial scores can be calculated using a set of artificial intelligence instructions, and wherein the initial scores estimate a likelihood of an event or a clinical status. In some aspects, the method can include calculating the data quality index based at least in part on the one or more initial scores, wherein the data quality index indicates a reliability of the patient score. The method can also include providing the data quality index to an output device.

In some examples, the initial scores can estimate a likelihood of a complication or an adverse risk for the patient. In some aspects, the one or more input data streams can include at least one vital measurement from a vital data stream, at least one patient characteristic, at least one lab measurement value, or a combination thereof. In some examples, the at least one feature can include a minimum value, a maximum value, a standard deviation value, a median value, a mean value, or any combination thereof, wherein the at least one feature is determined based on the one or more data input streams within a predetermined time period.

In some aspects, the set of artificial intelligence instructions can implement a neural network, the neural network comprising one or more relative values or a feature importance for the at least one feature. In some examples, the method can include determining a measurement threshold for the at least one feature, the measurement threshold representing a number of values used to calculate the at least one feature. In some aspects, the method can include identifying that the number of values for the at least one feature is below the minimum threshold. In some examples, the method can include calculating a data availability score for the at least one feature and calculating a data unavailability rank for the one or more input data streams with at least one missing feature, wherein the data unavailability rank is based at least in part on the data availability score and the relative value for each missing feature of the one or more data input streams. The method can also include generating a recommendation output indicating a request for one or more unavailable input data streams or one or more unavailable measurements for an input data stream associated with one or more missing features based on the data unavailability rank.

In some aspects, the method can include calculating an estimate of improvement in the data quality index following the addition of an unavailable input data stream. In some examples, the method can include identifying one or more missing features with the data availability score above a first threshold value, the one or more missing features to be requested for an input data stream with the data unavailability rank above the first threshold value. In some aspects, providing the data quality index can include transmitting the data quality index to an external device, displaying the data quality index, and generating an alert based on the data quality index, wherein the alert is provided by the external device.

In some examples, the data quality index can be displayed with a patient score value indicating a likelihood that the patient is experiencing the complication or the adverse risk. In some aspects, calculating the data quality index further can include calculating a variance value based on the one or more initial scores and determining the data quality index based at least in part on the variance value and a set of variance thresholds.

In some aspects, a system for generating a data quality index can include a processor that can identify a distribution for each of one or more features for a patient population. The processor can also detect one or more input data streams for a patient from a medical device, wherein the one or more input data streams comprise at least one vital measurement from a vital data stream, at least one patient characteristic, at least one lab measurement value, or a combination thereof. In some examples, the processor can receive a request to generate a patient score and the data quality index for the patient with a set of artificial intelligence instructions and the one or more input data streams and determine that at least one feature for the one or more input data streams is unavailable for the patient. The processor can also select one or more imputed values from the distribution for each of one or more features for the patient population and calculate one or more initial scores using the one or more imputed values from the distribution as input for the at least one feature that is unavailable, the initial scores calculated using a set of artificial intelligence instructions, wherein the initial scores estimate a likelihood of an event or a clinical status In some examples, the processor can also calculate the data quality index based at least in part on the one or more initial scores, wherein the data quality index indicates a reliability of the patient score. The method can also provide the data quality index to an output device.

In some aspects, a computer-readable medium for generating a data quality index can include a plurality of instructions that, in response to execution by a processor, cause the processor that can identify a distribution for each of one or more features for a patient population and detect one or more input data streams for a patient from a medical device, wherein the one or more input data streams comprise at least one vital measurement from a vital data stream, at least one patient characteristic, at least one lab measurement value, or a combination thereof. The instructions can also cause the processor to receive a request to generate a patient score and the data quality index for the patient with a set of artificial intelligence instructions and the one or more input data streams, determine that at least one feature for the one or more input data streams is unavailable for the patient, and select one or more imputed values from the distribution for each of one or more features for the patient population. The instructions can also cause the processor to calculate one or more initial scores using the one or more imputed values from the distribution as input for the at least one feature that is unavailable, the initial scores calculated using a set of artificial intelligence instructions, wherein the initial scores estimate a likelihood of an event or a clinical status. Furthermore, the instructions can cause the processor to calculate the data quality index based at least in part on the one or more initial scores, wherein the data quality index indicates an accuracy of the one or more initial scores, wherein calculating the data quality index comprises calculating a variance value based on the one or more initial scores and determining the data quality index based at least in part on a set of variance thresholds and the variance value. In some examples, the instructions can cause the processor to provide the data quality index to an output device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present examples will be better understood from reading the following description of non-limiting embodiments, with reference to the attached drawings, wherein below:

FIG. 1 schematically shows an example collaborative healthcare system.

FIG. 2 shows an example display device displaying a communication thread occurring on a communication channel of the collaborative healthcare system.

FIG. 3 shows an example display device displaying a dashboard of the collaborative healthcare system.

FIG. 4 shows an example display device displaying the dashboard of FIG. 3 including display of a portion of the communication thread of FIG. 2.

FIG. 5 shows an example display device displaying a collaborative interface.

FIG. 6 shows an example display device displaying another example dashboard of the collaborative healthcare system.

FIG. 7 shows a process flow diagram of an example method for generating a data quality index for a patient score.

FIG. 8 shows a process flow diagram of an example method for ranking unavailable input data streams and unavailable features.

FIG. 9 is a flow chart illustrating an example method for facilitating communication within a collaborative healthcare system.

FIG. 10 shows an example non-transitory computer-readable medium for generating a data quality index, a ranking of unavailable input data streams, or a combination thereof.

### DETAILED DESCRIPTION

### Introduction

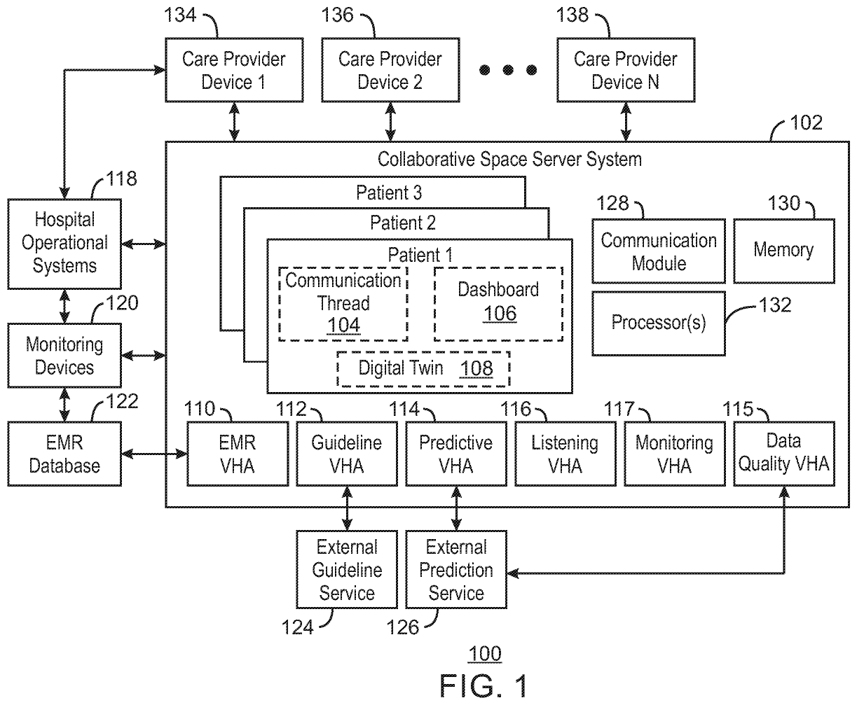

Embodiments of the present disclosure will now be described, by way of example, with reference to the FIGS. 1-10, which relate to various embodiments of a collaborative healthcare system that facilitates communication among care providers of a patient (which may be collectively referred to as a care provider team) and also utilizes machine and/or other deep learning models (e.g., in the form of virtual healthcare assistants) to perform certain patient monitoring and diagnostic activities. The collaborative healthcare system includes patient-specific communication channels that include communication thread-dashboard pairs to facilitate communication among the care providers and virtual healthcare assistants (also referred to as bots) on the communication thread while also graphically providing relevant patient care information (current vital signs, trends, medical history, etc.) to the care providers via the dashboard and/or communication thread. The techniques herein include generating a data quality index to display along with the information in the patient-specific communication channels, such as a patient score indicating a likelihood that a patient may experience an adverse condition. The data quality index can be provided with a patient score, in some examples, to indicate a reliability of the patient score when the patient score is generated with incomplete input data streams.

The virtual healthcare assistants may function as information retrievers, data monitors, predictors, and more to assist the care providers. The virtual healthcare assistants may provide requested patient data (e.g., fetch data from an electronic medical record), detect changes in patient state and alert the care providers of the changed state (e.g., by detecting that a patient vital sign has reached a condition relative to a threshold), and provide care guidelines, suggested diagnostic tests, and diagnoses to the care providers. The virtual healthcare assistants may be trained to communicate using natural language including medical language, thereby allowing for care providers to communicate with the virtual healthcare assistants in the same manner as other care providers.

Each communication channel may be specific to a given patient in a given acute care facility or other medical facility or healthcare setting (e.g., hospital, urgent care facility, or nursing home). A communication channel may be initiated upon admission of the patient to the medical facility. Each care provider of the patient may be joined to the communication channel, thereby allowing collaboration and communication among all care providers (e.g., doctors, nurses, and/or specialists such as radiologists) of the patient. The one or more virtual healthcare assistants may also be joined to the communication channel. Communication occurring on the communication channel may be in the form of text messages, rich media, and/or other forms, thereby allowing care providers to view graphs of patient medical trends, medical images, and so forth. Messages sent and received on the communication channel may be saved at a central location as a communication thread, allowing care providers to access prior conversations on the channel. For example, if a virtual healthcare assistant detects a change in a patient condition that indicates potential health issues, such as high blood pressure, the virtual healthcare assistant may note the high blood pressure and alert the care provider(s) via the communication thread. The blood pressure may be displayed via the patient dashboard along with the alert. A care provider may view the blood pressure measurement by selecting the alert in the communication thread. Later, the care provider may select a graphical display of the alert in the dashboard in order to launch the portion of the communication thread in which the blood pressure alert was issued.

The dashboard and communication thread may be viewable from a variety of client devices, including but not limited to a provider client device (such as a monitor in a nurse's station) and a provider mobile device (such as a tablet or smart phone). Thus, care providers may have access to relevant data and assistance from the virtual healthcare assistants from virtually any allowed location within the medical facility, and even off-site locations in some examples.

Further, the collaborative healthcare system described herein may facilitate flexible presentation of aggregate patient medical information, to enable care providers to review multiple, different pieces of medical data related to a patient with a single request. For example, a care provider may request, using natural language that is input via the communication thread, to view two, three, or more different patient medical parameters, such as heart rate, blood pressure, blood oxygen level, etc., over time.

In another example, a care provider may request, using natural language that is input via the communication thread, to view a summary of a patient medical condition, such as a pain summary, fever summary, etc. A virtual healthcare assistant may process the natural language input to determine the user request and then generate a list of patient medical parameters that are associated with the patient medical condition. The list of patient medical parameters may include currently monitored patient vital signs, medicine that has been administered to the patient, diagnostic imaging results for the patient (e.g., x-ray results), care provider-driven patient state assessments (e.g., alertness, skin color), lab test results, and so forth, each related to the patient medical condition. The virtual healthcare assistant may retrieve each patient medical parameter from the patient's electronic medical record, assemble the list, and then output the list for display via the communication thread.

In each of the above examples of the displayed aggregate medical information (e.g., the single graph of multiple plots of different medical data and the summary of the patient medical condition), each individual piece of medical information that is included in the displayed aggregate medical information may be individually viewable via various interfaces, such as via the communication thread, via the dashboard, and/or via a traditional care provider interface to the patient's electronic medical record. However, by providing the ability to present all of the medical information requested by the care provider in an aggregate form, the desired medical information may be presented with only a single request from the care provider, which may reduce the time required to view the medical information versus navigating through multiple menu layers/search inputs to view all of the desired information individually. Further, even though the medical information may be individually viewable via the other interfaces disclosed above, additional insight into the patient's condition may be missed if the care provider has to view separate graphs of the patient medical parameters. For example, if a care provider were to view individual graphs of heart rate and medication administration timing, the care provider may not realize that the patient's heart rate correlates with the timing of when the medication is administered. By providing a single graph of multiple plots of different medical data, correlation between different medical parameters/events may be more easily detected, while reducing user effort required to access the information. Further, the dashboard, where some but not all possible patient medical information may be viewed, may remain easy to navigate and view by limiting the information that may be viewed via the dashboard while still providing care providers the ability to access desired information via the communication thread.

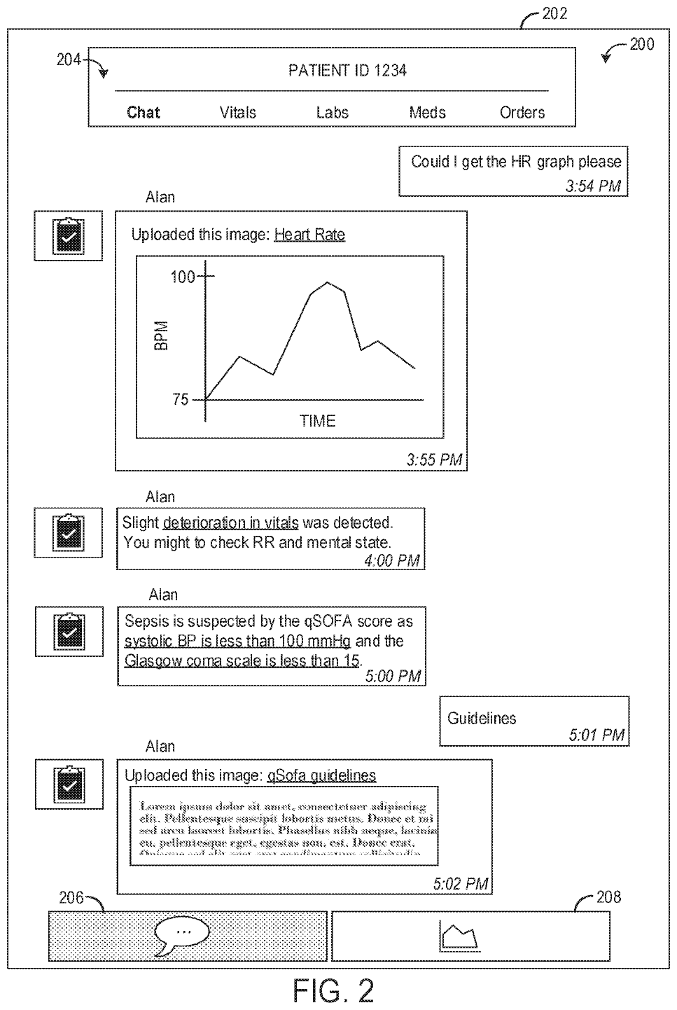

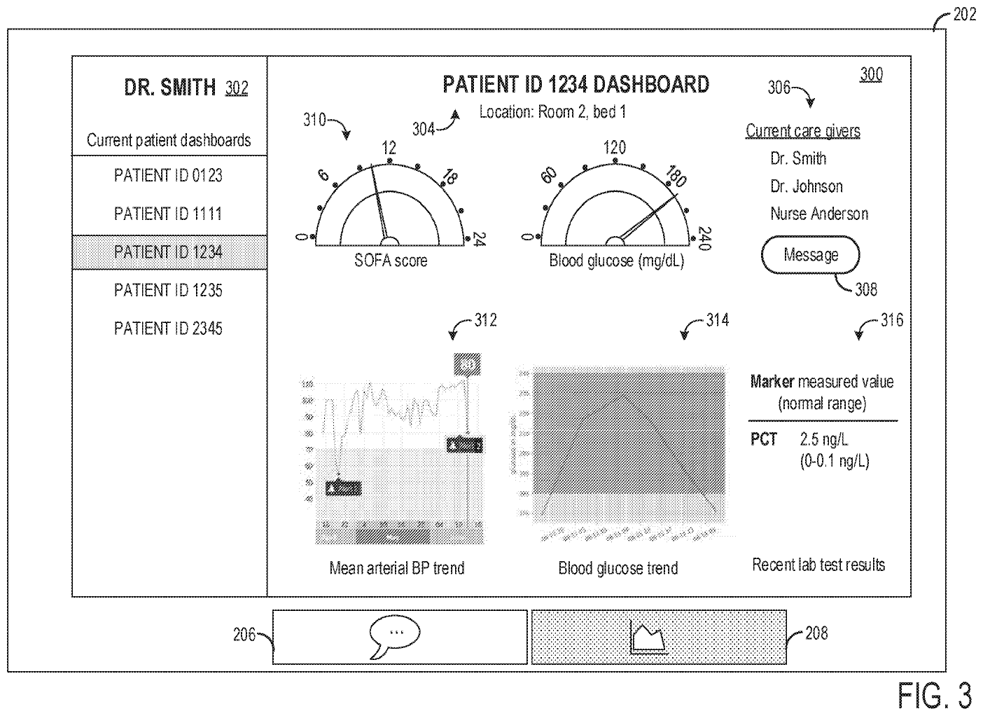

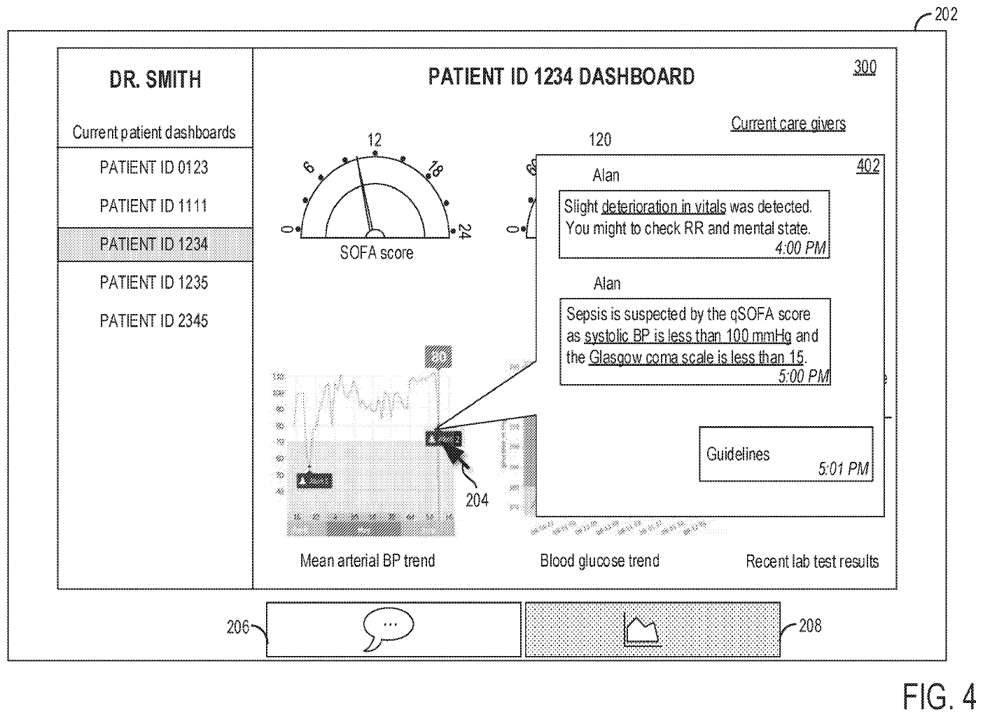

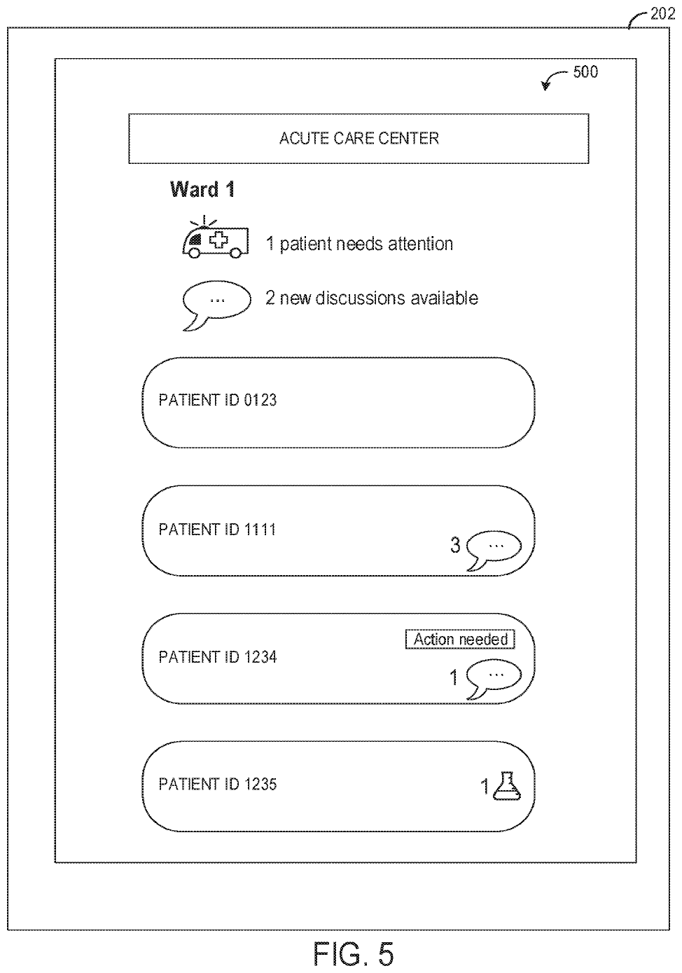

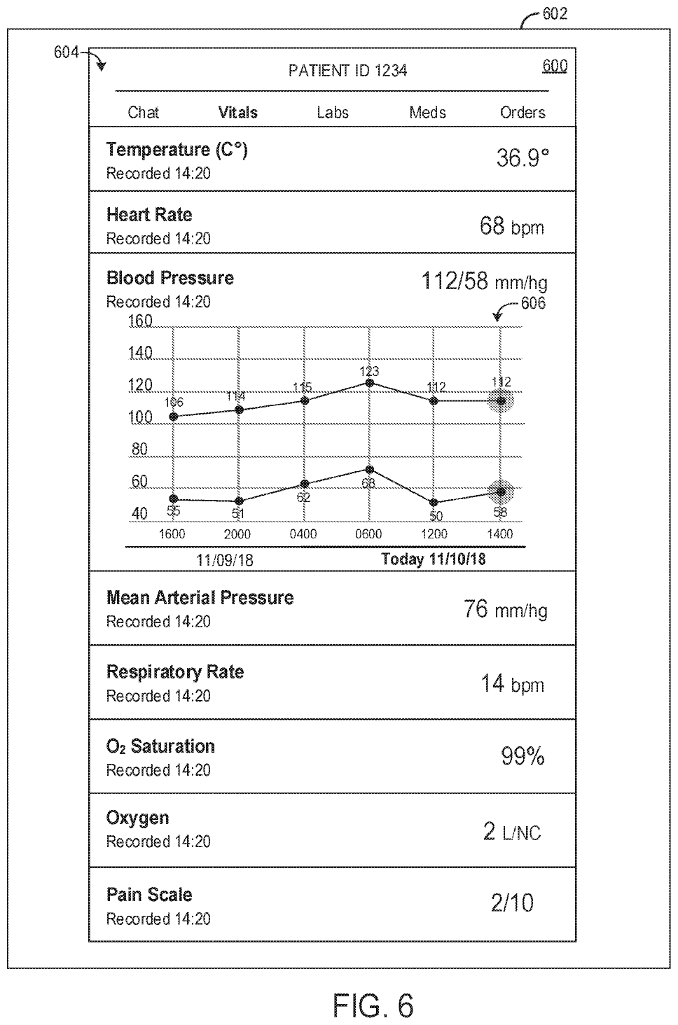

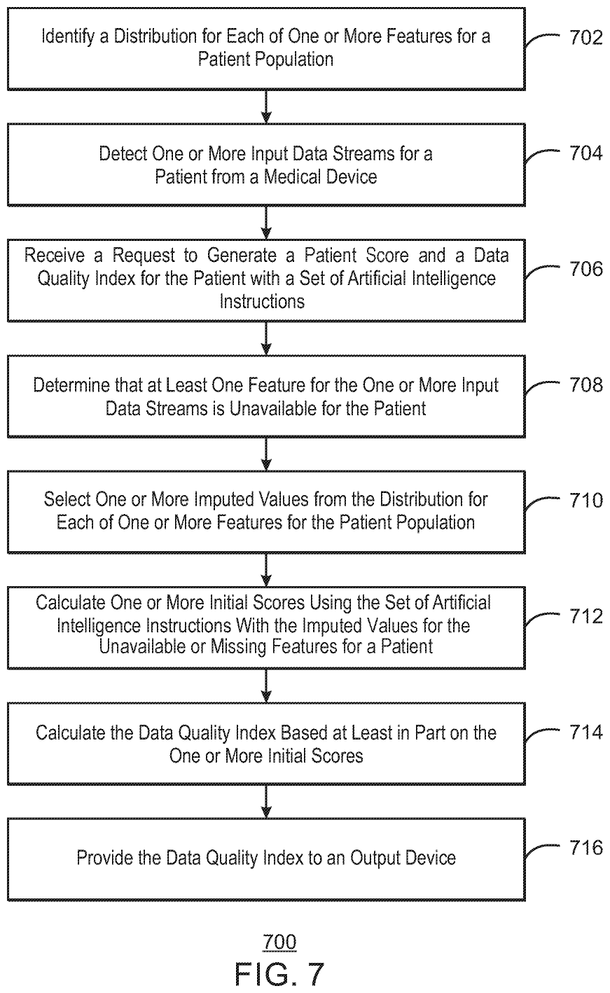

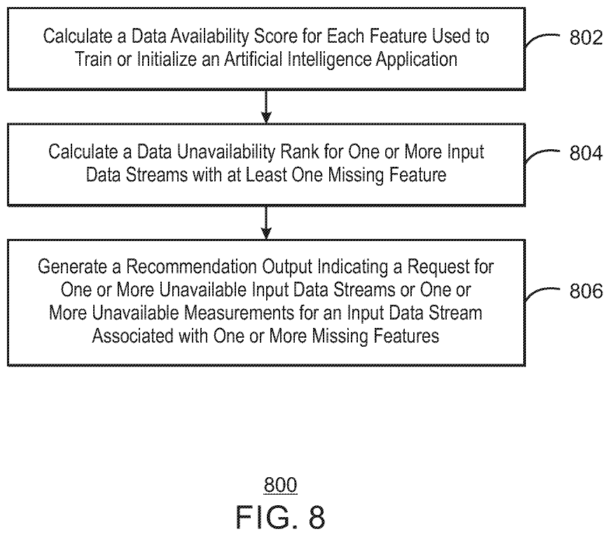

An example collaborative healthcare system is shown in FIG. 1. The collaborative healthcare system may be included in or associated with a medical facility and may include a communication channel comprising a communication thread and a dashboard for each admitted patient of the medical facility. The collaborative healthcare system may further include one or more virtual healthcare assistants. Communication may occur on a communication channel in the form of a communication thread (e.g., of text and/or rich media messages) between care providers of the patient and the one or more virtual healthcare assistants, as shown in FIG. 2. Patient-specific medical information may be displayed to the care providers and/or other users via a dashboard. As shown in FIG. 3, the dashboard may be launched in response to a first selection of a link on the communication thread. The dashboard may be configured to display alerts output by the one or more virtual healthcare assistants, and the alerts may be selectable to launch a portion of the communication thread occurring on the communication channel, as shown in FIG. 4, or a full version of the communication thread. Additionally, a preview of the dashboard may be launched in response to a second selection of a link on the communication thread. Further, a collaborative system interface, as shown in FIG. 5, may be displayed on a suitable display device in order to allow a user to select a communication channel or dashboard to view. In some examples, the dashboard may take on a different visual form, as shown in FIG. 6, and a user may navigate between the dashboard and communication thread using a menu that also allows the user to access additional information pertaining to the patient (e.g., medications, lab tests, etc.), as shown in FIG. 6. FIGS. 7 and 8 illustrate example techniques for generating data quality indices and ranking missing input data streams or input parameters that would improve the accuracy of the patient scores.

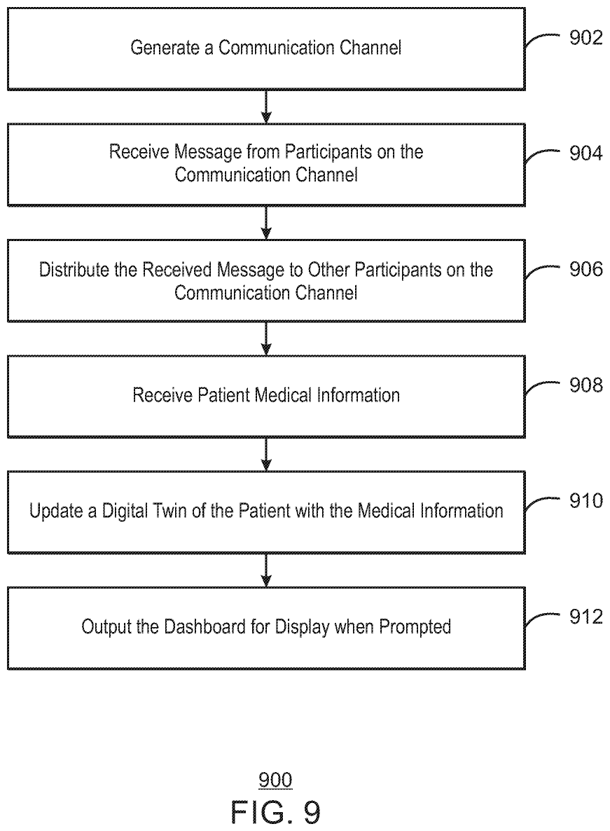

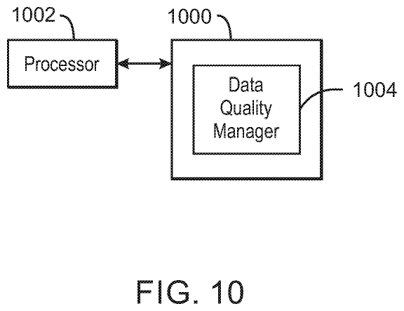

The communication thread-dashboard pairs may be generated and accessed according to the method illustrated in FIG. 9. The virtual healthcare assistants may provide assistance to the care providers of the patient by retrieving medical information of the patient from an electronic medical record and generating aggregate views of the medical information. FIG. 10 illustrates a non-transitory machine-readable media that can implement the methods of FIGS. 7, 8, and 9.

Techniques Described Herein

In some examples, a patient score can be generated, calculated, or otherwise obtained for each patient. The patient score can indicate a likelihood that a patient will experience an adverse condition. In some examples, the patient score is calculated or determined with an artificial intelligence application, such as a neural network, among others. The artificial intelligence application can be initialized or configured to generate the patient score based on any number of input data streams or input parameters such as a heart rate, a temperature, a glucose measurement, and the like. However, the input parameters for the artificial intelligence application may not be available for each patient. In some examples, the data set for each input data stream may be too small or there may not be any data values for some input data streams. The techniques herein can generate or calculate an estimated patient score for each patient and determine a data quality index for each estimated patient score. The data quality index can indicate if an estimated patient score is reliable or unreliable. For example, the data quality index can indicate if the techniques for generating the estimated patient score using imputed data values result in an unreliable output value.

The technical effect of generating a data quality index can enable care providers to determine the reliability of the patient score indicating if a patient is at risk of an adverse complication. In some examples, a system can train a machine learning technique to determine the likelihood of the risk of an adverse condition for a patient using a set of input data streams or input parameters. The present techniques have a technical advantage of enabling an artificial intelligence application or a machine learning technique to provide a data quality index to a care provider when input parameters or features are unavailable. For example, the techniques described herein can enable generating a likelihood of an adverse event for a patient using a subset of the input parameters or features used to train an artificial intelligence application or a machine learning technique and providing a data quality index that indicates the uncertainty of the provided likelihood value or estimated patient score due to missing inputs. The present techniques can also reduce the data storage and processing time of a medical system by generating a calculation of an adverse risk for a patient with a machine learning technique with missing input parameters rather than retraining the machine learning technique using a subset of input parameters. This can prevent the generation and initialization of multiple machine learning applications, which reduces processing time and data storage for the medical system.

FIG. 1 schematically shows an example collaborative healthcare system 100 that may be implemented in medical facility such as a hospital. Collaborative healthcare system 100 may include a collaborative space server system 102. Server system 102 may include resources (e.g., memory 130, processor(s) 132) that may be allocated to store and execute a communication thread, a dashboard, and a digital twin for each of a plurality of patients. For example, as shown in FIG. 1, a communication thread 104, dashboard 106, and digital twin 108 are stored on server system 102 for a first patient (patient 1); a plurality of additional communication threads, dashboards, and digital twins may be stored on server system 102, each corresponding to a respective patient (patient 2 up to patient N).

As explained above, the communication thread 104 may facilitate communication among a care provider team (which may include multiple care providers that are each providing care to the patient (e.g., patient 1)) as well as one or more virtual healthcare assistants (explained in more detail below). Messages sent on the communication thread 104 may be saved and may be accessible via the dashboard 106 (and the dashboard may be accessible via the communication thread). Further, the patient medical information, including medical history, current state, vital signs, and other information, may be entered to the digital twin 108, which may be used to gain situational awareness, clinical context, and medical history of the patient to facilitate predicted patient states, procurement of relevant treatment guidelines, patient state diagnoses, etc.

Communication occurring on communication thread 104 may be displayed on one or more suitable display devices associated with a respective care provider device and/or medical facility administration device. Likewise, dashboard 106 may be displayed on the one or more display devices. As shown in FIG. 1, a plurality of care provider devices, from a first care provider device 134, a second care provider device 136, and on up to an nth care provider device 138, may be communicatively coupled to server system 102. Each care provider device may include a processor, memory, communication module, user input device, display (e.g., screen or monitor), and/or other subsystems and may be in the form of a desktop computing device, a laptop computing device, a tablet, a smart phone, or other device. Each care provider device may be adapted to send and receive encrypted data and display medical information, including medical images in a suitable format such as digital imaging and communications in medicine (DICOM) or other standards. The care provider devices may be located locally at the medical facility (such as in a nurses station or in the room of a patient) and/or remotely from the medical facility (such as a care provider's mobile device).

When viewing communication thread 104 and/or dashboard 106 via a display of a care provider device, a care provider may enter input (e.g., via the user input device, which may include a keyboard, mouse, microphone, touch screen, stylus, or other device) that may be processed by the care provider device and sent to the server system 102. In examples where the user input is a message to be sent to other care providers and/or one or more virtual healthcare assistants, the message may be sent to the server system 102, where the message may be saved as part of the communication thread 104 and then the server system 102 may send the message to other verified participants on the communication channel (e.g., the other care providers and/or one or more virtual healthcare assistants that are joined to the communication channel). In examples where the user input is a selection of a link or user interface control button of the dashboard, the user input may trigger display of the communication thread, trigger progression to a desired state of the dashboard (e.g., trigger display of desired patient medical information), trigger updates to the configuration of the dashboard, or other actions.

The collaborative space server system 102 may be communicatively coupled to hospital operational systems 118. The hospital operational systems 118 may store and/or control a variety of hospital-, care provider-, and patient-related information, including but not limited to patient admission information (including date of admission and location of the patient within the medical facility), patient care protocols and workflows, and care provider information including which care providers are monitoring/treating which patients. Further, the hospital operational systems 118 may be communicatively coupled to a plurality of monitoring devices 120, an electronic medical records (EMR) database 122 (described in more detail below), and one or more of the care provider devices. The monitoring devices 120 may include traditional medical devices monitoring respective patients, such as pulse oximeters, heart rate monitors, blood glucose monitors, and ECGs, as well as microphones, cameras, and other devices. The monitoring devices 120 may send output directly to the server system 102 and/or may send output to the hospital operational systems 118, EMR database 122, and/or one or more care provider devices. For example, a plurality of monitoring devices monitoring patient 1 may be configured to send output to the server system 102 and the server system 102 may be configured to send some or all of the data output by the monitoring devices to a care provider device (such as care provider device 134). Further, in some examples, server system 102, hospital operational systems 118, and/or EMR database 122 may receive diagnostic imaging information obtained from one or more imaging modalities, such as ultrasound, CAT, MRI, X-ray, etc.

The hospital operational systems 118 may direct creation of and control access to each communication thread and dashboard. For example, when a patient is admitted, the hospital operational systems 118 may associate the patient with an identifier (e.g., an identification code) and notify the collaborative space server system 102 to generate a communication channel for that patient. When a care provider is assigned to assist in management/treatment of the patient, the hospital operational systems 118 may notify the collaborative space server system 102 to join that care provider to the patient's communication channel (the care provider may also be associated with an identifier which may be used to identify the care provider and appropriately distribute messages sent and received on the channel). In this way, the hospital operational systems 118 may control who has access to patient information. In some examples, hospital operational systems 118 and/or server system 102 may control levels of accessibility to patient information depending on the location of a care provider device (e.g., devices located at the medical facility may have access to more patient information than devices located remotely from the medical facility). Additional information about the hospital operational systems 118 is presented below.

Collaborative space server system 102 may further store instructions for (e.g., in memory 130) and be configured to execute (e.g., via processor(s) 132) a plurality of virtual healthcare assistants (VHAs). As shown, collaborative space server system 102 includes an electronic medical record (EMR) VHA 110, a guideline VHA 112, a predictive VHA 114, a listening VHA 116, and a monitoring VHA 117. The VHAs may be realized as several VHAs each for a different purpose, as described herein, various groups of VHAs (e.g., a the guideline VHA 112 and predictive VHA 114 may be combined into one VHA that is configured to both diagnose or predict patient state and output relevant guidelines), or as one overall VHA, which represents all the different attributes that will be hereby elaborated. All activations of VHAs by human care providers may be performed by using natural language including medical language, either by text or by voice.

EMR VHA 110 is configured to retrieve patient information from an electronic medical record database, such as EMR database 122, and present the retrieved data via the communication thread and/or dashboard. For example, a care provider may send a request to the EMR VHA 110, through the communication channel, for a particular piece of patient medical history saved in an EMR of the patient. The EMR VHA 110 may receive the request and determine, from the natural language of the text, that the piece of patient medical history has been requested. The EMR VHA 110 may obtain the piece of medical history from EMR database 122. The EMR VHA 110 may then send the piece of medical history to the care provider in the form of a message on the communication thread 104. In some examples where the requested piece of medical history is also saved in the digital twin 108, EMR VHA 110 may be configured to retrieve the medical history from the digital twin 108.

EMR database 122 may be an external database accessible by EMR VHA 110 via a secured hospital interface, or EMR database 122 may be a local database (e.g., housed on a device of the hospital). EMR database 122 may be a database stored in a mass storage device configured to communicate with secure channels (e.g., HTTPS and TLS), and store data in encrypted form. Further, the EMR mass storage device is configured to control access to patient electronic medical records such that only authorized healthcare providers may edit and access the electronic medical records. An EMR for a patient may include patient demographic information, family medical history, past medical history, lifestyle information, preexisting medical conditions, current medications, allergies, surgical history, past medical screenings and procedures, past hospitalizations and visits, etc.

Thus, the EMR VHA 110 serves as a connection to the EMR database. The EMR VHA may interpret questions by the human care providers regarding the patient and allows querying of the EMR database for relevant information regarding the patient (e.g. “what was the average systolic blood pressure in the last four hours?” or “show me the trend of the O2 saturation”). Queries can implicitly relate to the patient's status or medical history. The EMR VHA 110 also allows EMR-generated alerts to be formatted and sent into the patient communication thread (in a configurable manner either by a “setting” option or by voice command, such as telling it, e.g., “don't show me this again”). The EMR VHA 110 may also serve as a drug safety alerting system (including allergies, drug-to-drug relations, etc.) and may be thus connected to a relevant medical knowledgebase.

Guideline VHA 112 is configured to retrieve relevant care guidelines from an external guideline service 124. Guideline VHA 112 may be prompted, via communication occurring on communication channel, to retrieve care guidelines. For example, a care provider may explicitly request care guidelines for a given condition, such as sepsis, on the communication thread and guideline VHA 112 may query external guideline service 124 in response to the explicit request. In other examples, guideline VHA 112 may determine implicitly that care guidelines for a given patient condition are being requested and/or may be helpful. For example, guideline VHA 112 may parse communication on the communication thread 104 (e.g., between one or more care providers and/or a suitable VHA) to determine that guidelines are being requested (e.g., rather than receiving an explicit request for the guidelines, guideline VHA 112 may determine that two care providers are discussing guidelines and may retrieve the guidelines without being requested to do so). In a further example, guideline VHA 112 may determine, from patient vital signs (e.g., output by the one or more monitoring devices 120), digital twin 108, and/or other sources that a patient may be undergoing a given condition (e.g., high heart rate) and may automatically obtain guidelines for treating the condition.

External guideline service 124 may be a remote service accessed via a network, or external guideline service 124 may be a local service executed on a computing device of the hospital. The care guidelines obtained from external guideline service 124 may be preconfigured by protocols and guidelines that are specific to the medical facility that the collaborative space server system 102 services. Further, external guideline service 124 may include differential diagnoses trees that guideline VHA 112 may access to determine potential diagnoses based on a patient condition or state.

For example, with regards to the patient's state and medical history as search terms, e.g., if a diabetic patient has a high sequential organ failure assessment (SOFA) score and high glucose levels, specific guidelines will be queried without additional query terms, or alternatively the external guideline service may be queried by specifying specific guidelines. In other words, the guideline VHA may enter specific search terms to the guideline service based on patient state and symptoms (e.g., diabetes, SOFA score of five, glucose level of 190 mg/dL) to obtain one or more potential diagnoses and/or guidelines, or the guideline VHA may specifically ask for guidelines for a given condition (e.g., sepsis). The guideline VHA may also serve as a source for generating reminders for treatments that are part of a care protocol or to keep track of what decision-driving tests have been completed and what are still needed to complete the protocol. A change in patient status may be a trigger for automatic notification of relevant guidelines. The guideline VHA may also be used to plan a trajectory for the patient, of both disease progression and a care path. A patient trajectory may be determined based on the combined trajectories of vital signs, laboratory test results, or other data for that specific patient. In defining a patient trajectory, the guideline VHA may assist care providers to adjust care pathways or to stay the course and give early warning if the patient deviates from the planned trajectory.

Predictive VHA 114 is configured to retrieve predictions of future patient states from an external prediction service 126. Predictive VHA 114 may detect and issue alerts on relevant changes in the patient's state (e.g., small but worrying changes in vital signs, changes in qSOFA score). Predictive VHA 114 may also predict future events (e.g., a prediction of sepsis being developed in the coming four hours) by connecting to external prediction service 126. Predictive VHA 114 may query external prediction service 126 with search terms indicating current and/or past patient state (e.g., blood pressure trend, glucose level trend, etc.). If prediction service 126 outputs a possible future condition, the predictive VHA 114 may send an alert into the communication thread, as text, and may provide supplemental information regarding the alert. The predictive VHA 114 may also track the response of human care providers as reflected in the communication channel or in the EMR orders registry. The predictive VHA 114 may obtain patient data from the EMR and different online monitoring devices 120 (ECG, cameras, etc.) as represented in the digital twin.

In some examples, the predictive VHA 114 can retrieve, request, or otherwise obtain data quality information from a data quality VHA 115. The data quality VHA 115 can calculate a data quality index based at least in part on one or more initial scores, such as patient scores that indicate if a patient has an increased risk of developing sepsis, high blood pressure, or the like. The data quality index can indicate an accuracy of the initial scores when the initial scores are calculated with missing or imputed data values. In some examples, the data quality VHA 115 alone or in combination with the predictive VHA 114 can provide the data quality index to the care provider devices 134, 136, and 138 at a predetermined time interval, continuously, or in response to a query. The techniques for calculating the data quality index and ranking missing input data streams are described in greater detail below in relation to FIGS. 7 and 8.

Listening VHA 116 is configured to monitor communication on the communication thread 104 as well as actual human voice communication to obtain/infer various information related to the patient. In doing so, listening VHA 116 serves as a monitor, by listening to the events in the patient's surroundings including medical staff conversations and patient input (from moaning to speech). The monitored conversations/inputs may be used to record the patient's status (for EMR/digital twin) or to infer clinician reasoning (e.g., the listening VHA may catch an order to prescribe a certain antibiotic by a doctor, and understand an infection is suspected). The listening VHA 116 may receive output from one or more microphones positioned in proximity to the patient, for example, in order to monitor the conversations and inputs.

Monitoring VHA 117 is configured to receive output from the monitoring devices 120 and may track a patient condition or state based on the received output. In some examples, monitoring VHA 117 may present the received data via the communication thread and/or dashboard. For example, a care provider may send a request to the monitoring VHA 117, through the communication channel, for a particular piece of patient monitoring data, such as current heart rate. The monitoring VHA 117 may receive the request and determine, from the natural language of the request, that the patient medical data has been requested. The monitoring VHA 117 may obtain the patient medical data from the relevant monitoring device of the monitoring devices 120. The monitoring VHA 117 may then send the medical data to the care provider in the form of a message on the communication thread 104. In some examples, monitoring VHA 117 may be configured to save the medical data at the digital twin 108. Further, medical data received by monitoring VHA 117 may be displayed via the dashboard. In some examples, monitoring VHA 117 may obtain patient medical data only in response to a request from a care provider. In other examples, additionally or alternatively, monitoring VHA 117 may obtain medical data from the monitoring devices 120 independently of care giver request, and may output requested medical data when a care giver requests the data and/or when the received medical data is detected (by the VHA) as being abnormal, having changed, or otherwise indicative of an urgent patient state. In some examples, monitoring VHA 117 may be configured to provide received medical data to predictive VHA 114 and/or guideline VHA 112 in order to predict a future patient state based on current patient medical data and/or retrieve relevant care guidelines based on current patient medical data.

The VHAs may be configured to receive messages from human care providers and utilize natural language processing to determine what information is being conveyed in the messages. For example, the VHAs may utilize natural language processing to determine if a message received on the communication channel includes a request for patient medical information, and if so, determine what medical information is being requested. The VHAs may also be configured to process medical information of the patient (e.g., vital signs, medical history, current symptoms) received from the patient EMR, the monitoring devices, the care providers, and/or other sources and determine which parameters of the medical information may be used (e.g., entered into the guideline or prediction service) to determine a patient state (such as determine the likelihood the patient is experiencing a certain condition, such as sepsis). The VHAs may execute machine learning models (e.g., deep learning or other machine learning models such as neural networking) that are trained to understand medical terminology. Further, the machine learning models may be configured to learn updates or modifications to the models in an ongoing manner in a patient and/or care provider specific manner. For example, a predictive VHA may execute a deep learning model that is trained to determine that low blood pressure may be a symptom of relevance that should be entered into a prediction service or diagnosis tree, but then may be trained for a specific patient that low blood pressure for that patient is benign and may have less relevance.

The models may be trained in a suitable manner. In a first example, the models may be rule-based assistants that are configured with a set of answers for predetermined, likely questions. When a VHA receives a question, the VHA may be configured to output an answer from the set of answers. In a second example, the models may include directed acyclic graphs (DAG) of states, each of which include rules for how to react and how to proceed to various questions. However, such VHAs may only be configured to respond when there is a clear indication of the user intent (e.g., the user presses on a button “obtain heart rate”) and entities (answer to “please provide the patient's date of birth” with a date).

Thus, the VHAs described herein may include artificial intelligence and be adapted to handle natural language which is a way to take human input and map it to intent and entities. The VHAs may be adapted to hold a state and map the state with (intent, entities) to an actionable API. The mapping may be performed by teaching machine learning models by providing the models with examples of such mappings. If a VHA is autonomous, the VHA may include a prediction or other mechanism that may trigger the VHA to initiate communication. The VHAs may also be configured to vary their reactions to make the VHAs more human like (this may also be performed by providing examples to a machine learning training algorithm).

Further, the training mechanism utilized may be specific for different VHAs. For example, the listening VHA (and the natural language processing engines of the other VHAs) may execute deep networks trained for natural language with medical language. This may be combined with taxonomies from the medical domain. The EMR VHA and the guideline VHA may receive the output (intent and entities) from the listening VHA and/or the respective natural language processing engine and map the output to queries. The VHAs may be trained by having examples of the best results of existing queries. The predictive VHA may be trained on its own clinical task. For example, if the predictive VHA is to predict if a patient will survive early release from an intensive care unit, then the predictive VHA may be trained on data of patients that were in the ICU and were released at different stages.

Additional VHAs may be included on the server system, such as VHAs specific to a patient state. Such an example may include a sepsis VHA that may only be joined to a patient communication channel when that patient is undergoing or at risk of developing sepsis. The sepsis VHA may be trained to specifically predict sepsis, obtain treatment guidelines for sepsis, suggest optimal lab tests to diagnose sepsis and/or monitor sepsis progression, and/or suggest treatment options for sepsis. Other VHAs may include a patient comfort VHA (e.g., a VHA configured to detect or predict patient pain, discomfort, hunger, or other symptoms not necessarily indicative of a particular medical condition but which care providers may want to be notified of to improve patient comfort), a communication VHA (e.g., that parses communication from care givers and facilitates sharing of information among the VHAs), and/or other VHAs. Further, various configurations of VHAs not disclosed above are within the scope of this disclosure, such as related VHAs being grouped into a single VHA (e.g., the monitoring and EMR VHAs being combined as one medical data VHR). For example, a single VHA may be trained for all of the above-described VHA possible skills.

A global view of multiple or all patient communication thread-dashboard pairs may be provided via to one or more of the care provider devices and the hospital operational systems 118. For example, the choice of the specific thread/dashboard pair to access may be controlled by an access application executing on collaborative space server system 102 that allows to a user to view all the relevant patients (for example, communication thread-dashboard pairs for all the patients being treated/monitored in a nurses station may be accessed on a workstation at the nurses station, or communication thread-dashboard pairs for all the patients being treated/monitored by a given care provider may accessed by that care provider on his or her mobile device). In some examples, alerts and important events within all the relevant communication channels will be signified in the global view. The choice to go into a specific communication channel may be made by a user picking the patient in the global view (or by an explicit voice command), but may be also be automated using automatic mechanisms which may detect the position of the care provider in respect to a patient (such as via BLUETOOTH® when entering a patient's proximity or based the context of a detected discussion).

The access application may allow export of only specific widgets (such as the blood pressure graph of a patient) of a communication thread and/or dashboard, or may allow more compound parts (such as a patient dashboard or a portion of the thread) to selected external applications and/or devices. For example, as explained above, devices located off-site of the medical facility may only be allowed access to some of the patient medical data, and the access application may control which patient medical data is viewable outside of the medical facility.

A management application executed on hospital operational systems 118 and/or collaborative space server system 102 may allow an administrator to update the care team that has access to a patient's communications channel, as described above. The management application may include an interface for configuring hospital specific protocols and care guidelines. The management application may also aggregate information from the communication channels to be used to predict needs for hospital operations, presenting forecasts for capital, disposable, and human assets based on aggregate acuity or disease statistics. Moreover, analytics of the information on the communication channel may be employed to improve the system and its predictors.

Collaborative space server system 102 includes a communication module 128, memory 130, and processor(s) 132 to store and execute the communication channel-dashboard pairs, digital twins, and VHAs, as well as send and receive communications, graphical user interfaces, medical data, and other information.

Communication module 128 facilitates transmission of electronic data within and/or among one or more systems. Communication via communication module 128 can be implemented using one or more protocols. In some examples, communication via communication module 128 occurs according to one or more standards (e.g., Digital Imaging and Communications in Medicine (DICOM), Health Level Seven (HL7), ANSI X12N, etc.). Communication module 128 can be a wired interface (e.g., a data bus, a Universal Serial Bus (USB) connection, etc.) and/or a wireless interface (e.g., radio frequency, infrared, near field communication (NFC), etc.). For example, communication module 128 may communicate via wired local area network (LAN), wireless LAN, wide area network (WAN), etc. using any past, present, or future communication protocol (e.g., BLUETOOTH™, USB 2.0, USB 3.0, etc.).

Memory 130 one or more data storage structures, such as optical memory devices, magnetic memory devices, or solid-state memory devices, for storing programs and routines executed by processor(s) 132 to carry out various functionalities disclosed herein. Memory 130 may include any desired type of volatile and/or non-volatile memory such as, for example, static random access memory (SRAM), dynamic random access memory (DRAM), flash memory, read-only memory (ROM), etc. Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

One or more of the devices described herein may be implemented over a cloud or other computer network. For example, server system 102 is shown in FIG. 1 as constituting a single entity, but it is to be understood that server system 102 may be distributed across multiple devices, such as across multiple servers.

While not specifically shown in FIG. 1, additional devices described herein (care provider device 134, care provider device 136, and care provider device 138, hospital operational systems 118, monitoring devices 120, EMR database 122, external guideline service 124, external prediction service 126) may likewise include user input devices, memory, processors, and communication modules/interfaces similar to communication module 128, memory 130, and processor(s) 132 described above, and thus the description of communication module 128, memory 130, and processor(s) 132 likewise applies to the other devices described herein. As an example, the care provider devices (e.g., care provider device 134) may store user interface templates in memory that include placeholders for relevant information stored on server system 102. For example, care provider device 134 may store a user interface template for a patient dashboard that a user of care provider device 134 may configure with placeholders for desired patient information. When the dashboard is displayed on the care provider device, the relevant patient information may be retrieved from server system 102 and inserted in the placeholders. The patient information may include current patient vital signs, VHA alerts, desired patient state trends, or other information, as explained in more detail below. The user input devices may include keyboards, mice, touch screens, microphones, or other suitable devices.

FIG. 2 shows an example communication thread 200 of a patient-specific communication channel, and as such may be a non-limiting example of communication thread 104. Communication thread 200 may be displayed on a display device 202. Display device 202 may include a screen on which the communication thread is displayed and may be coupled to and/or included as a part of a computing device, such as care provider device 134. Communication thread 200 may be displayed in response to a user request to display the communication thread. For example, the user (e.g., a care provider) may access a collaborative system interface that includes a global view of all communication threads and dashboards the user is authenticated to participate in (which may include all patients at the medical facility the care provider is attending to) and may select a desired communication thread to view. An example collaborative system interface 500 is shown in FIG. 5. Collaborative system interface 500 may be displayed on display device 202 or other suitable device and may include all patients admitted to a specific unit or ward of a medical facility. As shown, collaborative system interface 500 includes identifying information specifying the medical facility (“acute care center”) and relevant unit (“ward 1”) of the medical facility, and further includes links to patient-specific communication threads and dashboards for the patients in that unit of that medical facility. However, in other examples, the patients shown via collaborative system interface 500 may specific to a certain care provider.

Collaborative system interface 500 may include a notification section whereby the user viewing collaborative system interface 500 may be notified of urgent patient conditions, active communication channel discussions, lab test results, and other information. For example, collaborative system interface 500 includes a notification section that shows that one patient requires attention (e.g., due to deteriorating vital signs) while two new discussions are available.

Collaborative system interface 500 further includes links to patient communication thread-dashboard pairs. For example, FIG. 5 shows links to communication thread-dashboard pairs for patient ID 0123, patient ID 1111, patient ID 1234, and patient ID 1235. As explained above, each patient may be assigned an identifier that may be used to identify the patient on the communication thread and dashboard. In other examples, other mechanisms for identifying the patient may be used, such as location (e.g., bed 2 in room 4) and/or actual patient name. Additional patient links may be viewed by scrolling the interface. Each patient link may include notifications where relevant. For example, the link for patient ID 1111 includes a notification that three new messages are available to be viewed on the communication channel for that patient. The link for patient ID 1234 includes a notification that action is needed (e.g., due to high blood pressure or other significant vital sign being detected, which will be explained in more detail below) as well as a notification that one new message is available on the communication channel for that patient. In some examples, when a lab test result for a patient is available, the link for that patient may include a notification of an available lab test result, such as the notification displayed in the link for patient ID 1235. In examples, care providers may be notified of available lab test results through the communication channel for the patient, and thus the notification may include a notification that a new message is available.

Selection of a patient link may launch the communication thread or dashboard for that patient. For example, selection of the link for patient ID 1234 may launch the communication thread 200 for patient ID 1234, shown FIG. 2 and explained in more detail below.

Returning to FIG. 2, communication thread 200 may include an identification header 204 that identifies the patient being discussed/monitored via the communication thread. In the illustrated example, communication thread 200 is specific to patient ID 1234. In the illustrated portion of communication thread 200, communication is occurring between a care provider (e.g., Dr. Smith) and a virtual healthcare assistant that, in the illustrated example, has a human persona and as such includes a human name (Alan). Communication thread 200 is being viewed by Dr. Smith, although any authenticated/approved user may view communication thread 200. As shown by the first message from the top, Dr. Smith is requesting medical information relating to the patient from the VHA by asking, in natural language, for a heart rate graph (“Could I get the HR graph please”). In response, the VHA sends an image of the patient's heart rate graph, which may be obtained or assembled from the patient's EMR data. The image of the heart rate graph is viewable in the communication thread and may also be selected via suitable user input to view in a different form, such as via the patient dashboard.

At a later time (e.g., 4:00 PM), the VHA outputs an alert/notification in the communication thread indicating a change in patient status, herein a deterioration in vitals. The alert is accompanied by a suggested course of action that a care provider may take, including checking respiratory rate and mental state. The VHA issues another alert at 5:00 PM indicating that sepsis is suspected based on a quick SOFA score (qSOFA), owing to low systolic blood pressure and a low Glasgow coma scale. The alerts issued by the VHA may include links to the patient dashboard, for example, allowing a user to select a link to launch the dashboard and view the medical data relating to the alerts. For example, the alert “systolic BP is less than 100 mmHg” is shown in underline, indicating a link to additional information is available. A user may select the link via a suitable input, such as via a mouse click, touch input, or voice command. In some examples, selecting a link with a first selection (e.g., a double click) may launch the patient dashboard (as shown in FIG. 3). Selecting a link with a second selection (e.g., a single click or a hover) may launch a preview where only the patient's blood pressure graph is shown.

In response to the alert regarding the potential sepsis, Dr. Smith asks for guidelines at 5:01 PM. Because the VHA had immediately previously issued the alert regarding the possible sepsis due to the qSOFA score, the VHA may assume that the guidelines being requested by Dr. Smith include guidelines for sepsis based on a qSOFA score. In response, the VHA retrieves guidelines from an external guideline service relating to qSOFA scores and outputs the guidelines into the communication thread. As shown, only a portion of the guidelines are displayed in the communication thread. By selecting the link (the underlined “qSofa guidelines”), the user may be taken to a different interface where the full guidelines are displayed, or the full guidelines may be displayed over the top of the still-displayed communication thread.

While not shown in FIG. 2, communication thread 200 may include a search box/functionality where a user may search for past messages on the communication thread. For example, a user may enter a command (by voice or text) requesting that all messages related to the patient's heart rate be displayed. Also displayed on display device 202 is a communication thread button 206 and a dashboard button 208. In FIG. 2, the user is viewing the communication thread 200 occurring on the communication channel. Hence, the communication thread button 206 is highlighted. To switch to the dashboard for patient ID 1234, the user may select the dashboard button 208. Additionally or alternatively, the user may request to view a vital signs interface by selecting a link within the identification header 204 (e.g., by selecting “vitals”). The vital signs interface, which is discussed in more detail below with respect to FIG. 6, may serve as an additional or alternative dashboard, wherein additional medical information of the patient may be displayed. The user may request to view additional interfaces via the identification header 204, such as a labs interface, where lab results may be displayed, a meds interface, where medicine dosage, timing, and other information may be displayed, and an orders interface, where testing orders (such as diagnostic imaging tests, lab tests, or other tests) may be displayed.

As explained above, FIG. 2 shows communication between a care provider and a virtual healthcare assistant (“Alan”), where Alan provides all the information requested by the care provider as well as provides alerts/notifications. In this way, Alan is performing the functions described above with respect to FIG. 1 ascribed to the predictive VHA, EMR VHA, and guidelines VHA. It is to be understood that in some examples, each of the different VHAs may interact with the communication thread individually.

FIG. 3 shows an example dashboard 300 that may be displayed on display device 202 or other suitable device. Dashboard 300 may be displayed in response to a user input to the communication thread 200, for example by selecting a link within medical information displayed in communication thread, as explained above, or in response to selection of the dashboard button 208. However, dashboard 300 may be displayed in response to other inputs, such as in response to a user input selecting the dashboard from the collaborative system interface of FIG. 10 that includes a global view of multiple communication threads and dashboards. Additionally, FIG. 3 shows a side bar 302 displayed along with dashboard 300 showing patient dashboards for the patients Dr. Smith is currently attending. User input to the side bar may launch a different dashboard, for example Dr. Smith may select to view each of the currently available dashboards to quickly assess the status of each patient.

Dashboard 300 may be configured to display patient medical information based on the current patient state and user-configured settings. For example, a dashboard for a patient that is being treated at the medical facility for pneumonia may be configured to display different medical information than a dashboard for a patient that is being treated at the medical facility for a stroke. In some examples, when a patient is admitted at the medical facility, a dashboard may be generated automatically for the patient based on the reason of admittance (e.g., pneumonia), thereby including the most relevant patient medical information for the patient's condition, such as blood oxygen level and respiration rate. A user may also configure which medical data to view via the dashboard, for example a doctor attending to the patient may choose to view heart rate rather than respiration rate.

The medical information that is displayed on the dashboard may be obtained from one or more monitoring devices currently monitoring the patient, such that the medical information is displayed on the dashboard in a real-time (or near real-time) manner. Additionally or alternatively, the medical information that is displayed on the dashboard may be obtained from the patient's EMR, the digital twin associated with the patient, and/or the communication thread. As explained above, one or more VHAs may obtain patient medical information from the patient's EMR, the monitoring devices, guideline services, or other sources and include the obtained medical information as a message in a communication thread on the communication channel. To view the medical information in greater detail, the user may select the medical information from the communication thread, where the medical information may then be displayed in the dashboard.

Additional information may also be displayed via the dashboard, such as patient information (location, demographics, medical history), care provider information (such as which doctors, nurses, and/or other care providers are attending to the patient), and a timeline of selected or relevant messages from the communication thread. For example, the most recent alerts may be displayed as a timeline on the dashboard.

Referring to dashboard 300 as an example, patient information 304 is displayed at the top of the dashboard, including patient identification and location. Care provider information 306 is also displayed in dashboard 300, including current care providers for the patient. Additionally, a user interface control button 308 is shown that, when selected, may allow the care provider viewing dashboard 300 to view and interact with the communication thread.

Dashboard 300 further includes real-time medical information indicators 310. As shown, the indicators 310 include a SOFA score and blood glucose level, depicted as gauge charts with respective needles that move to indicate current SOFA score and blood glucose relative to a range of possible SOFA scores and blood glucose levels. While not shown in FIG. 3, the gauge charts may include color coding for quick determination of normal, intermediate, and high scores/levels, for example. The gauge charts shown are exemplary in nature and patient medical information may be shown in other forms.

Dashboard further includes medical history trends, including a first graph 312 depicting mean arterial blood pressure trend (e.g., blood pressure as a function of time) and a second graph 314 depicting blood glucose trend (e.g., blood pressure as a function of time). The medical history trends shown in FIG. 3 may be displayed on the dashboard in response to a request from a user (e.g., in response to a care provider selecting a link to patient medical history from a communication thread), due to a preconfigured dashboard setting, or other suitable trigger. For example, as shown in FIG. 2, the VHA issued an alert at 5:00 PM that included reference to patient blood pressure in the form of a link. When the link is selected (e.g., via cursor 204), the dashboard 300 may be displayed showing the first graph 312 of the patient's blood pressure trend.

Dashboard 300 further includes a recent lab test results section 316, where the results from recent lab tests may be displayed. For example, the user may have selected a link to available procalcitonin (PCT) test results displayed as part of a communication thread, which may result in display of dashboard 300. Via the recent lab test results section 316, the user (care provider) may be notified that the PCT test for that patient is relatively high and thus sepsis is confirmed or suspected.

As explained earlier, one or more of the virtual healthcare assistants may be configured to monitor patient vital signs, via the output from the monitoring devices, the information stored in the digital twin, or other source. If a vital sign (or other health parameter) meets a predetermined condition, the one or more virtual healthcare assistants may be configured to output an alert to notify the one or more care providers attending the patient that patient follow-up may be needed. The alerts may be included in the communication thread, as discussed above. Additionally or alternatively, the alerts may be displayed on the dashboard. As shown, first graph 312 includes two alerts, each alert issued when mean arterial blood pressure dropped below a threshold, such as 80 mmHg, or trended in an unexpected way, such as five consecutively decreasing values which may or may not be below the 80 mmHg threshold. Selection of an alert may trigger display of a portion of the communication thread occurring on the communication channel where the alert was referenced.

Thus, as shown in FIG. 4, in response to user input selecting the second alert displayed on the dashboard 300 (e.g., the “alert 2” box) via cursor 204, a portion 402 of the communication thread 200 shown in FIG. 2 is displayed over dashboard 300. The portion 402 displayed may include only the portion of the communication thread that references the medical information that triggered the alert, and may also include additional messages around the message referencing the alert, in order to place the alert in context. In this way, a user may be able to quickly determine what else may have occurred around the time the alert was issued, determine if attending care providers administered treatment, or determine other relevant information. The portion 402 may not include the most recent messages in the communication thread, in some examples. Further, a user may not have access to the full communication thread when viewing the portion, and may not be able to interact (e.g., send messages) with the communication channel. Thus, a different selection on the dashboard may enable a user to view the full communication thread.

Additionally or alternatively, when viewing the portion of the communication thread, the user may scroll to view other portions of the communication thread or may enter another input to the portion of the communication thread to enable viewing of the full version of the communication thread. Alternatively, instead of showing a snippet from the communication channel, the full version of the communication thread may be displayed, with the focus point being the point in the communication channel that references the alert (which may enable the user to look before and after that point of the thread if desired). In another example, only the snippet of the communication thread may be displayed and if the snippet is selected, the full version of the communication thread may be displayed. In this way, either automatically or upon a further user input, the use may be able to interact with the communication thread (e.g., send a message via the communication thread).

Thus, the collaborative healthcare system shown in FIG. 1 may generate communication channel-dashboard pairs for each patient associated with the collaborative healthcare system. The collaborative healthcare system may include one or more computing devices, such as the care provider device 134. The computing device may include a display screen, and the computing device may be configured to display on the screen a dashboard. The dashboard may include patient medical information, such as diagnostic lab test information (e.g., lab test results, pending lab tests that have been ordered but not yet fulfilled, status updates for pending lab tests, and so forth). The computing device may additionally be configured to display on the screen an alert related to the patient medical information. For example, as shown in FIG. 3, dashboard 300 may be displayed on a screen of a computing device (e.g., display device 202, which may be a screen of a computing device such as care provider device 134). Dashboard 300 may display patient medical information, such as the graph of the blood pressure trend of the patient (e.g., first graph 312) and recent lab results. The displayed medical information may include an alert, such as alert 2 shown on first graph 312.

The alert may be selectable to launch a communication thread between a care provider and a virtual healthcare assistant. For example, as shown in FIG. 4, selection of alert 2 launches a communication thread between a care provider (Dr. Smith) and a virtual healthcare assistant (Alan). The selection of the alert enables a portion of the communication thread that references the displayed patient medical information to be seen within the communication thread. For example, FIG. 4 shows that in response to selection of alert 2, a portion 402 of the communication thread 202 (shown in FIG. 2) is displayed. The portion 402 includes reference to patient blood pressure, which is also displayed on the patient dashboard. Further, the alert may be displayed on the dashboard (at least initially) while the communication thread is in an un-launched state. For example, the alert may be displayed on dashboard 300 without display of the communication thread, e.g., while the communication thread is un-launched. In some examples, the full communication thread may be displayed rather than just a portion, with the full communication thread focused at the portion that references the patient blood pressure. According to some embodiments, the communication thread and the dashboard may both be displayed simultaneously on the display device.

In this way, the computing device provides a specific manner of displaying a limited set of information (e.g., the portion of the communication thread that specifically references medical information displayed on the dashboard) to the user, rather than using conventional user interface methods to display a generic index/list on a computer that may require the user to step through multiple menus and/or lists of communications and alerts to find the relevant portion of communication regarding the medical information. The dashboard interface-communication thread link disclosed herein may be advantageous because it avoids a user having to scroll around and switch views multiple times to find desired data/functionality, thereby preventing drilling down through many layers to get the desired data/functionality which may be slow, complex, and difficult to learn. The disclosed dashboard interface-communication thread link may improve the efficiency of using the computing device by bringing together the portion of the communication thread most relevant to the user (as it relates to the displayed medical information) and the dashboard actually displaying the medical information, allowing the user to view the most relevant information on the communication thread without actually opening up the communication thread. The speed of a user's navigation through various views and windows may be improved because the disclosed link between the dashboard and the communication thread saves the user from navigating to the communication thread from the dashboard, opening the communication thread up, and then navigating within the communication thread to enable the portion of interest to be seen or a function of interest to be activated.

Thus, the collaborative healthcare system shown in FIG. 1 may generate communication thread-dashboard pairs for each patient associated with the collaborative healthcare system. The collaborative healthcare system may include one or more computing devices, such as the care provider device 134. The computing device may include a display screen, and the computing device may be configured to display on the screen a communication thread. The computing device may additionally be configured to display on the screen a dashboard that can be reached directly from the communication thread. For example, as shown in FIG. 2, the communication thread may include a link that when selected launches a dashboard, such as the dashboard 300 shown in FIG. 3.

The communication thread displays communication between a care provider and a virtual healthcare assistant, and the communication thread includes medical information of a patient. At least a portion of the displayed medical information is selectable to launch the dashboard and enable the selected medical information to be seen within the dashboard. For example, referring to FIG. 2, the communication thread includes a link referencing patient medical information (herein, lab test results), and selection of the link launches some or all of the dashboard. The dashboard includes display of the patient medical information included in the link (e.g., the lab test results). In an example, selection of the link may launch a full version of the dashboard, as shown in FIG. 3. In another example, selection of the link may launch only a portion of the dashboard. The communication thread may be displayed while the dashboard is in an un-launched state, at least initially. For example, FIG. 2 shows the communication thread being displayed without display of the dashboard, and thus the dashboard may be unlaunched until the link the communication thread is selected.

In this way, the computing device provides a specific manner of displaying a limited set of information (e.g., the dashboard that specifically includes medical information referenced in the communication thread) to the user, rather than using conventional user interface methods to display a generic index/list on a computer that may require the user to step through multiple menus and/or lists of communications and alerts to find the relevant medical information. The communication thread-dashboard interface link disclosed herein may be advantageous because it avoids a user having to scroll around and switch views multiple times to find desired data/functionality, thereby preventing drilling down through many layers to get the desired data/functionality which may be slow, complex, and difficult to learn. The disclosed communication thread-dashboard interface link may improve the efficiency of using the computing device by bringing together the medical information most relevant to the user (via the dashboard) and the communication thread referencing the medical information, allowing the user to view the most relevant medical information discussed on the communication thread without actually accessing an electronic medical record or separate interface where patient monitoring data may be displayed. The speed of a user's navigation through various views and windows may be improved because the disclosed link between the communication thread and dashboard saves the user from navigating to an electronic medical record database, opening the database up, and then navigating within the database to enable the medical information of interest to be seen or a function of interest to be activated.

As mentioned previously, an identification header may be displayed with the communication thread and may include patient identification information and user interface control buttons via which a user may enter input selecting to view a different interface related to that patient. As an example, the identification header may include a vitals button, and user selection of the vitals button (e.g., via a touch input) may cause a vital signs interface for that patient to be displayed. FIG. 6 shows an example of a vital signs interface 600 that may be displayed on a display device 602. Display device 602 may include a screen on which the vital signs interface is displayed and may be coupled to and/or included as a part of a computing device, such as care provider device 134 shown in FIG. 1.

Vital signs interface 600 includes an identification header 604, similar to the identification header described above with respect to FIG. 2. Vital signs interface 600 further includes a plurality of patient medical parameters. The plurality of patient medical parameters may include a plurality of vital signs measured by one or more patient monitoring devices, which may be stored in the patient's EMR and/or as part of the patient's digital twin. The plurality of vital signs may include temperature, heart rate, blood pressure, mean arterial pressure, respiratory rate, O2 saturation, and oxygen level, as shown, and/or may include additional or alternative vital signs. The plurality of patient medical parameters may further include assessed patient states, which may be ascertained by one or more care providers and entered into the patient's EMR. The assessed patient states may include pain (as shown), alertness level, cognition, appearance, etc.

For each medical parameter displayed on the vital signs interface 600, a name of the medical parameter may be displayed along with the most recently-recorded value for that parameter and the time/date at which the value was recorded. For example, the first vital sign displayed from the top of the vital signs interface includes temperature with a value of 36.9° C. recorded at 14:20. For some, or all, of the patient medical parameters displayed on the vital signs interface, user selection of that medical parameter may result in additional information being displayed, such as a trend graph for that parameter that may include some or all of the measured values for that parameter over a given duration. For example, as shown, a user has selected the blood pressure parameter, e.g., by entering a touch input to the blood pressure parameter displayed on the interface. As a result, a blood pressure trend graph 606 is displayed (e.g., the blood pressure section may expand downward to accommodate the graph, shifting all other parameters below it downward). The blood pressure trend graph 606 includes six blood pressure values measured in the past 24 hours and plotted as a function of time (as each blood pressure measurement includes systolic and diastolic pressure, two curves are shown, one for systolic and one for diastolic). User deselection of the blood pressure parameter (e.g., by selecting it again) may cause the blood pressure region to collapse back to its original size and displayed information, resulting in the blood pressure trend graph not being displayed. Similar trend graphs may displayed upon user selection of any of the displayed medical parameters.

The medical parameters that are displayed as part of the vital signs interface may be selected in a suitable manner. In one example, the user may customize which parameters are displayed on the vital signs interface, whether globally for all patients that the user interacts with or individually by patient. In other examples, an administrator (e.g., of the hospital) may determine which parameters are displayed. In still further examples, additionally or alternatively, the medical parameters included in a particular patient's vital signs interface may be based at least in part on the patient's diagnosed condition(s) and/or reason for admittance to the medical unit. For example, a vital signs interface specific to a patient that is diagnosed with pneumonia may have at least some different medical parameters than a vital signs interface specific to a patient undergoing a C-section.

The vital signs interface illustrated in FIG. 6 may be displayable in addition to the patient dashboard illustrated in FIGS. 3 and 4. In this way, both the dashboard of FIGS. 3 and 4 and the vital signs interface of FIG. 6 may be available to care givers as different interfaces for viewing select patient medical information. In other examples, the vital signs interface and the dashboard illustrated in FIGS. 3 and 4 may be alternative embodiments, e.g., the vital signs interface may be one example of how a dashboard may be configured and the dashboard of FIGS. 3 and 4 may be a different example of how a dashboard may be configured. In still other examples, the vital signs interface may be a “mobile interface” and the dashboard (such as the dashboard of FIGS. 3 and 4) may be a “standard interface,” such that the dashboard may be viewed when the care provider device is a desktop computer, laptop, large format monitor, etc., while the vitals interface may be displayed when the care provider device is a mobile device, such as a smartphone or tablet, or otherwise includes limited display area.

In some examples, the vital signs interface can include a patient score that indicates if a patient is likely to develop an adverse complication or condition, such as sepsis, among others. If the patient score is calculated with imputed values in place of missing input data or missing features, the vital signs interface can also include a data quality index that reflects the uncertainty of the prediction caused by the missing input. The data quality index can be displayed within the vital signs interface, the chat interface, the labs interface, the orders interface, the other interface, or the data quality index can be displayed within a separate interface. In some examples, additional information can be displayed along with the data quality index. For example, the additional information can include missing input data streams, missing features, and the like. In some examples, the additional information can include a ranking of the missing input data streams and missing features. The ranking can indicate the missing input data streams or missing features that would most improve the data quality index. Techniques for calculating the data quality index and the ranking of the missing input data streams and missing features are described below in relation to FIGS. 7 and 8.

FIG. 7 is an example method for providing a data quality index. In some examples, the method 700 can be implemented with any suitable computing device, such as the collaborative space server system 102 of FIG. 1, or the care provider devices 134, 136, and 138 of FIG. 1, among others.

At block 702, the method 700 can include identifying a distribution for each of one or more features for a patient population. In some examples, the features can include any suitable mathematical calculation, such as a minimum, a maximum, a mean, a median, or a standard deviation, among others, based on an input data stream. The input data streams can include any suitable set of data values for a vital sign for a patient, a set of data values for one or more laboratory tests for a patient, a set of data values for one or more patient characteristics, or a combination thereof. In some examples, any number of features can be calculated for each input data stream for a period of time.

The distribution for each feature of a patient population can include detecting at least one set of data values for each input parameter to be monitored for a patient population and calculating the distribution for each feature. For example, the distribution can be calculated for each vital sign, such as the heart rate, blood oxygen saturation (SPO2), or the like. In some examples, the distribution can include any number of data values for each feature or input parameter to form a curve defined by any suitable probability density function. Identifying the distribution for each feature can be performed on a sample set of patient data and used to enable selecting imputed data values as discussed in greater detail below.

At block 704, the method 700 can include detecting one or more input data streams for a patient from a medical device. The input data streams can include any number of vital sign data streams, laboratory data streams, patient characteristics, or the like. In some examples, the input data streams can include at least one vital measurement from a vital data stream, at least one patient characteristic, at least one lab measurement value, or a combination thereof. For example, the input data streams can include vital data such as a data set of time values for a heart rate of a patient, a temperature measurement of a patient, or a blood oxygen saturation (SPO2) measurement of a patient, among others. A patient characteristic can include a height of a patient, a weight of a patient, a known disease of the patient, and the like. The lab measurement values can include any suitable data values detected by a blood test or an imaging system, among others. For example, the lab measurement values can include a measurement of white blood cell count, a sodium measurement, a carbon dioxide measurement, a calcium measurement, a protein measurement, a globulin measurement, a glucose measurement, or a combination thereof.

In some examples, each input data stream can have one or more features calculated based on the data values from each input data stream. The features can include a minimum value, a maximum value, a standard deviation value, a median value, a mean value, or any combination thereof, wherein the features are determined based on the one or more data input streams within a predetermined time period.

At block 706, the method 700 can include receiving a request to generate a patient score and a data quality index for the patient with a set of artificial intelligence instructions and the one or more input data streams. The patient score can indicate a likelihood that a patient will experience an event or a change in clinical status. An event or change in clinical status can include the onset of an intervention, transfer to higher acuity care, or an adverse episode such as cardiac arrest or death. In some examples, the request can be detected by a virtual health assistant. For example, a request can be provided by a clinician, or any other suitable user using a virtual health assistant for a patient. In some examples, the data quality index can indicate the quality and completeness of available data streams used as input for further processing, such as for a patient score, indicating a likelihood that a patient may experience an adverse condition. For example, the data quality index can indicate the reliability of a patient score estimating that a patient may experience an adverse condition based on the input data streams and the features calculated based on the input data streams. In some examples, a request to generate a data quality index can be automatically generated each time a patient score is obtained, calculated, or received. The data quality index may be refreshed or recalculated after any suitable period of time.

In some examples, the request is received with a VHA (such as the EMR VHA, in examples where multiple VHAs are available), which can provide, obtain, or generate one or more graphs that include plots of heart rate, blood pressure, and oxygen saturation for a patient within a period of time. As another example, user input may include a request to view information associated with a data quality index, such as temperature, heart rate, blood pressure, and the like.

In some examples, the most recent temperature measurement, heart rate measurement, and blood pressure measurement that are stored in the patient's EMR (and/or digital twin) may be retrieved by the VHA. The parameters that are included in the list of parameters may be predefined by a user or by an administration official. For example, the user requesting the information (e.g., the care provider) may customize which input parameters are included in a summary accompanying the data quality index. In other examples, the medical facility may determine which input parameters, features, or a combination thereof, are included in each patient condition summary. The parameters that may be included in a summary include medical data obtained from monitoring devices (e.g., heart rate, blood pressure, temperature), assessed patient states (e.g., energy level, skin color, cognition), diagnostic imaging results, lab test results, and/or medications (e.g., dosage, timing), each of which are determined to relevant to a condition of a patient. Further, in some examples, the summary may include only the most recently-obtained values for each parameter. In other examples, the summary may include trends (e.g., graphs) for one or more of the parameters. Whether the summary includes the most-recently obtained values or the trends may depend on user preference, administration preference, type of parameters included in the summary, and/or the patient condition associated with the summary.

At block 708, the method 700 can include determining that at least one feature for the one or more input data streams is unavailable for the patient. For example, the number of data values for an input data stream can be below a minimum threshold that prevents calculating features. In some examples, the number of data values for calculating a feature may be 1, 2, 5, 10, 20, 50, or any other suitable number. For example, to determine a feature that indicates a mean value of a heart rate for a patient within a predetermined period of time, the minimum threshold can be 10 data values, or any other suitable number.

In some examples, each of the features for an input data stream can be missing or unavailable if the input data stream is unavailable. For example, the sensor data indicating a heart rate, SPO2 data, or any other vital sign information, lab measurement data, or patient characteristic information may not be available for calculating features. In some examples, any number of features can be missing or unavailable for one or more input data streams or input parameters. For example, a first feature can be missing from a first input data stream, such as a heart rate, and a second feature can be missing from a second input data stream, such as blood pressure. In some examples, each input parameter can be missing a different number of features. For example, a first input parameter or input data stream can be missing two features and a second input parameter or input data stream can be missing three features or more. The different input data streams can be missing a different number of features due to each feature using a different number of data values for calculation. For example, calculating a feature for a mean value or a median value may require more data values than a feature for a minimum value or a maximum value.

In some examples, the method 700 can include determining a measurement threshold for a feature, wherein the measurement threshold represents a number of values used to calculate the feature. The method 700 can also include identifying that the number of values for a feature is below the minimum threshold indicating that the feature is to be considered unavailable or missing.

At block 710, the method 700 can include selecting one or more values from the distribution for each of one or more features for the patient population for imputation. In some examples, the method 700 can include selecting any number of imputed values from the distributions corresponding to the missing features. For example, the method 700 can include randomly selecting 5, 10, 20, or any other suitable number of values from a distribution corresponding to heart rate values previously identified within a patient population. In some examples, the imputed values can be sampled from the distributions corresponding to missing features using any suitable technique or mathematical operation. For example, the imputed values can be sampled, selected, or otherwise identified from the distributions for the missing features.

In some examples, the method 700 can include determining a number of missing features, identifying a distribution corresponding to each of the missing features, and selecting any number of imputed values from each of the identified distributions corresponding to missing features. The number of imputed values selected from each distribution can be the same or the number of imputed values selected from each distribution can differ. For example, five imputed values may be selected from a distribution corresponding to a first missing feature and ten imputed values may be selected from a distribution corresponding to a second missing feature. The number of imputed values to select from each distribution can be a static predetermined value or the number of imputed values can be dynamically adjusted based on user input.

At block 712, the method 700 can include calculating one or more initial scores using the set of artificial intelligence instructions with imputed values for the unavailable or missing features for a patient. For example, the initial scores can be estimated patient scores representing a likelihood of a patient experiencing an adverse condition. The initial scores can be calculated using the one or more imputed values from the distribution as input for the at least one feature that is unavailable. In some examples, the set of artificial intelligence instructions can implement any suitable supervised learning technique, an unsupervised learning technique, a reinforcement learning technique, or the like. In some examples, a supervised learning technique can implement a neural network, among others, to identify a set of weight values to apply to any number of input data streams for calculating a likelihood of an adverse risk or complication of a patient. The neural network can be trained with any number of input data streams and any number of features that are calculated for each of the input data streams. In some examples, a feature importance or relative value of each feature is determined as a mean decrease in impurity caused by the feature for decision trees constituting a classifier. In some examples, one or more features for any number of the input data streams can be missing or unavailable if the vital sign data, laboratory data, or the like, are missing or unavailable for a patient. The neural network, or any other suitable machine learning technique, can calculate the initial scores by replacing the missing data values for each of the missing features with imputed values selected using techniques described above in relation to block 710.

In some examples, the method 700 can include calculating an initial score using each of the selected imputed values. The method 700 can also include calculating additional initial scores using any number of random values, or predetermined values, among others. The initial scores can represent estimates of patient scores based on a set of input data streams, features, or a combination thereof, used to train an artificial intelligence application or machine learning technique that calculates the patient score.

At block 714, the method 700 can include calculating a data quality index based at least in part on the one or more initial scores, wherein the data quality index indicates an accuracy of the patient score based on a combination of the initial scores. For example, the method 700 can include calculating a variance, a mean, a median, or any other suitable value based on the initial score values. In some examples, the method 700 can include calculating the data quality index as a maximum of (i) a half-width of a confidence interval, such as 95%, among others, of the initial score values and (ii) a difference between the patient score and the mean of the initial score values, among other values.

In some examples, the output provided to a care provider device can include a patient score (PS) that can be a numerical score or an alphanumerical score, among others, representing the physiological status of the patient, assessed as the statistical risk of the patient requiring cardiovascular or pulmonary intervention. The score range can be from 0 (stable, low risk of intervention needed) to 100 (unstable, high risk of intervention needed) or the score range can use any other suitable range or spectrum of scores. The data quality index can provide a quantitative representation of the degree of reliability in the patient score output given the specific set of inputs available for generating the patient score.

In some examples, the data quality index is determined by the variability of the classifier output or machine learning technique output, wherein the variability is caused by missing input parameters or features. The variability can be estimated by repeatedly replacing the missing inputs or missing feature values corresponding to input data streams with data values selected or identified from the input parameters' distributions over a training data set. In some examples, the variability can be estimated by calculating the resulting patient score (PS′) or output from a machine learning technique for each set of replaced or imputed data values. From the multiple obtained patient score values, a mean of the patient scores and the half-width (w) of a confidence interval can be calculated. In some examples, a data quality index can then be calculated as 100−max(|PS−mean(PS′)|,w), where PS represents the original Patient Score being evaluated.

At block 716, the method 700 can include providing the data quality index to an output device. In some examples, the method 700 can include determining any suitable number of thresholds that categorize the data quality index as reliable, moderate, unreliable, or questionable, among others. For example, an unreliable or low data quality index can indicate that the missing data values have a large effect on the artificial intelligence instructions and that the patient score being provided has a low confidence value. The unreliable data quality index values and the associated patient scores can be discarded in some implementations. In some examples, the high quality category corresponds to a high data quality index value, while a moderate quality category falls in between the unreliable or discarded values and the high quality values. In one example, the method 700 can include calculating a variance value based on one or more initial scores and determining the data quality index based at least in part on a set of variance thresholds and the variance value. For example, a higher variance value can indicate that the missing features or missing input data streams used to calculate the initial scores have a large effect that results in a data quality index that may be discarded or considered to be moderate quality. A lower variance score can indicate that the missing features or missing input data streams used to calculate the initial scores have a small effect that results in a data quality index that may be considered high quality and reliable. For example, the lower variance value can indicate that the imputed values used to calculate the initial scores resulted in consistent results and that the associated patient score can be used by a care provider device to assess a condition of a patient.

In some examples, the method 700 can include providing the data quality index by transmitting the data quality index to an external device, displaying the data quality index, and generating an alert based on the data quality index, wherein the alert is provided by the external device. For example, the alert can be provided by a care provider device or a hospital monitor, among other devices. In some examples, the data quality index can be provided to any suitable display device, mobile device, or the like. In some examples, the method 700 can be implemented by a remote computing device and the data quality index can be provided to a mobile device proximate a patient or within a hospital or facility treating the patient. In some examples, the data quality index is displayed adjacent to or with a patient score value indicating a likelihood that the patient is experiencing the complication or the adverse risk.

In some examples, the data quality index can be accompanied by any suitable alarm, alert, or the like, that is provided to any number of devices associated with the patient. The alarm or alert can result in an audio message, a text message, or a combination thereof, being provided by the devices associated with a patient. For example, the alarm or alert can result in the devices associated with a patient indicating a warning for sepsis, a high or low heart rate, a low blood oxygen saturation rate, or need for interventions, among others. In some examples, the alarm or alert can indicate insufficient input data for an input data stream or a feature.

In some examples, the method 700 can be implemented as a software as a service, or a cloud based service, that is executed by any number of remote servers. The data quality index, among other output, can be provided from the remote servers to devices, such as mobile devices, tablets, patient monitors, and the like, associated with a patient.

The process flow diagram of method 700 of FIG. 7 is not intended to indicate that all of the operations of blocks 702-716 of the method 700 are to be included in every example. Additionally, the process flow diagram of method 700 of FIG. 7 describes a possible order of executing operations. However, it is to be understood that the operations of the method 700 can be implemented in various orders or sequences. In addition, in some examples, the method 700 can also include fewer or additional operations. In some examples, if all features are available, the method 700 can include generating a patient score without generating the data quality index.

FIG. 8 illustrates a process flow diagram of an example method for ranking unavailable features. In some examples, the method 800 can be implemented with any suitable computing device, such as the collaborative space server system 102 of FIG. 1, the care provider devices 134, 136, and 138 of FIG. 1, among others.

At block 802, the method 800 can include calculating a data availability score for each feature used to train or initialize an artificial intelligence application. In some examples, the data availability score can indicate a number of data values available for each feature. For example, each feature may have 5, 10, 20, 50, or any other number of data values detected by sensors, user input, or a combination thereof. In some examples, the data availability score is based on a number of data values received or obtained for each feature and a data threshold. The data threshold can indicate a minimum number of data values to calculate each feature. For example, the data threshold can indicate that five heart rate values, or any other number, is the minimum threshold for determining a median heart rate within a period of time, a mean heart rate within a period of time, or any other suitable calculation or metric for a patient.

At block 804, the method 800 can include calculating a data unavailability rank for one or more input data streams with at least one missing feature. For example, the data unavailability rank can be calculated for a missing heart rate, a missing SPO2 value, a missing laboratory test, or a missing blood sugar value, among others. In some examples, the data unavailability rank is based at least in part on the data availability score and the relative value or feature importance for each missing feature of the one or more data input streams. The relative value or feature importance can be any suitable predetermined value or a value, such as a weighted value, determined by a machine learning technique. In some examples, the data unavailability rank can be calculated based on Equation 1 below:

$$\sum ( ( 1 - \frac{data availability actual}{data availability expected} ) \times feature importance ) \qquad Eq . ( 1 )$$

In some examples, the Eq. 1 can be applied to each feature for an input data stream and the results for each feature can be combined to indicate the data unavailability rank for an input data stream or input parameter. The data availability expected value can be any suitable predetermined value that represents a number of expected data values to be received, obtained, or otherwise acquired.

At block 806, the method 800 can include generating a recommendation output indicating a request for one or more unavailable input data streams or one or more unavailable measurements for an input data stream associated with one or more missing features. In some examples, the recommendation can be based on the data unavailability rank. For example, the recommendation can include a missing parameters list that includes a list of expected input parameters that are either missing or whose measurement frequency is suboptimal. The input parameters may have a smaller number of data values than expected, which can reduce or prevent accurately measuring, calculating, or obtaining feature values for the input parameters.

In some examples, the method 800 can include identifying one or more missing features with a data unavailability rank above a first threshold value. The method 800 can also include requesting to obtain or receive one or more missing feature values for an input data stream with the data unavailability rank above a threshold value. For example, the method 800 can include identifying that two features for a heart rate input data stream are unavailable and additional data values for the heart rate input data can be requested or obtained to enable the calculation of the two missing features. In some examples, the additional data values for missing features can be requested if the missing features have a data unavailability rank above a threshold value. The method 800 can also include calculating an estimate of improvement in the data quality index following the addition of an unavailable input data stream. For example, adding, obtaining, or otherwise receiving an input data stream can result in a data quality index value that corresponds to a more reliable patient score.

The process flow diagram of method 800 of FIG. 8 is not intended to indicate that all of the operations of blocks 802-806 of the method 800 are to be included in every example. Additionally, the process flow diagram of method 800 of FIG. 8 describes a possible order of executing operations. However, it is to be understood that the operations of the method 800 can be implemented in various orders or sequences. In addition, in some examples, the method 800 can also include fewer or additional operations.

FIG. 9 is a flow chart illustrating a method 900 for a collaborative healthcare system serving a medical facility, such as a hospital. Method 900 can be executed by a processor of a computing device (such as processor(s) 132 of server system 102 of FIG. 1) according to instructions stored on a non-transitory memory of the device (e.g., memory 130 shown in FIG. 1) in combination with the various signals received at the server system from components of the collaborative healthcare system (e.g., patient medical data signals from monitoring devices 120, communication from hospital operational systems 118, etc.) and signals sent from the server system to the care provider devices and/or other system components.

At 902, method 900 includes generating a communication channel including a communication thread and a dashboard for the patient. In order to generate the communication channel, verified care providers of the patient (e.g., as indicated by the notification from the hospital operational systems) and one or more virtual healthcare assistants (VHAs) may be joined to the communication channel, in some examples. The communication channel may facilitate text and/or rich-media based messages to be sent among all the verified care providers and VHAs that are joined to the communication channel. The one or more VHAs may include an EMR VHA, a guideline VHA, a predictive VHA, a listening VHA, a monitoring VHA and/or other VHAs. To join the channel, each VHA may receive a message that a new channel has been opened and the access application (e.g., executing on the server system 102) may add the VHAs to the eligible participants of the channel. Moreover, in some examples, not all available VHAs may be invited to all channels (e.g., a sepsis VHA may not be invited in a non-relevant case or the listening VHA may not be invited due to patient refusal to be monitored by recording).

Generating the dashboard may include configuring the dashboard based on the patient state and/or user settings, in some examples. As explained previously, a patient dashboard may be a graphical user interface that facilitates display of patient medical information, such as real-time vital signs, medical history, treatment plan, and/or other information. The dashboard may also include relevant/desired messages from the communication thread. Which medical information to display on the dashboard and in what format may be determined based on the patient state (e.g., current medical condition for which the patient is being treated) and/or on user settings, which may be configured by the end-viewer of the dashboard. In this manner, different patients may have different medical information displayed on different dashboards, and different care providers may view different medical information for the same patient, if desired.

At 904, method 900 includes receiving text- and/or rich-media-based messages from the participants on the communication channel, including care providers and VHAs. During the course of patient care, care providers may communicate with each other on the communication channel via messages of the communication thread to coordinate care, give care instructions, and/or confirm appropriate care is being carried out. Further, care providers may send requests to the VHAs via the communication thread for various information related to the patient care, including patient medical history, care guidelines, predicted future patient state, recommended lab tests, etc. Further still, VHAs may send notifications via the communication thread of changes in patient state, patient medical history, patient care guidelines, predicted future patient states, lab test status, etc. The messages sent from a care provider may be sent from a care provider device (e.g., device 134) and received at the server system via a suitable connection (e.g., wired or wireless, such as via the Internet). The messages sent from the VHAs may be generated by the VHAs, which may be stored and executed on the server system, the cloud, and/or a remote device. As used herein, messages may refer to any suitable information sent and received on the communication thread, including but not limited to text messages (entered via typing, touch, or stylus input, voice input, or automatically generated by a VHA), images, voice messages (e.g., recordings of voice input), and videos.

At 906, method 900 includes distributing the received messages to other participants on the communication channel and saving the received messages as a communication thread. Each message that is sent to the server system may be tagged with various identifiers that identify the sender as well as the patient communication thread to which the message pertains (e.g., the patient identifier). The server system may then send the message to other participants of the communication channel, e.g., the care providers and/or VHAs that did not send the original message, and save the message as part of a saved communication thread. The saved communication thread may then be viewed by other users at other times, retrieved in response to a user request to view some or all of the communication thread, etc. However, in some examples, the device from which the original message was sent (e.g., the care provider device) may send the message to all other participants on the communication channel, and thus the server system may not distribute the message to the other participants.

At 908, method 900 includes receiving patient medical information. The patient medical information may be received from one or more patient monitoring devices that are configured to measure patient state and condition, including sensors that measure vital signs (e.g., blood pressure, heart rate, and blood oxygen level), diagnostic imaging modalities, microphones in proximity to the patient, and so forth. Additionally, the patient medical information may be received from the communication thread. For example, two care providers may be messaging each other on the communication thread and exchanging information relating to the patient, such as visual information (e.g., skin pallor, redness, or yellowness) of the patient that may indicative of patient state. One or more of the VHAs may be configured to parse the message and determine that relevant medical information is being exchanged and then save the medical information as messages within the communication thread.

At 910, method 900 includes updating a digital twin of the patient with the medical information. The digital twin may be a digital replica/representation of the patient that is saved at the computing device (e.g., digital twin 108 saved on the server system 102). The digital twin may include patient demographic information, medical history, and other information to provide, to the extent possible, a simulation/representation of the current patient medical state. When new or updated medical information is received, the digital twin may be updated to reflect the most recent patient medical state. The digital twin may be accessed (e.g., by one or more of the VHAs) to retrieve patient medical information, predict future patient states (e.g., simulations may be performed using the information stored in the digital twin to determine the probability of the patient developing a certain condition), identify the most relevant lab tests to be conducted to diagnose a patient condition, and provide appropriate context when retrieving care guidelines.

In some examples, the method 900 can include updating the digital twin to reflect a current data quality index and a recommendation to obtain missing input data streams, missing features, or a combination thereof. For example, the digital twin can store a series of data quality index values for a patient that can be accessed based on timestamp data or a query for data quality index values generated based on particular input data streams, among others.

In some examples, the data quality index and the recommendation for missing input data streams or features can be forwarded or shared between any number of devices within a network. For example, a first care provider device can forward a data quality index or a recommendation for obtaining missing input streams or features to a second care provider device or a hospital monitor, among others.

At 912, method 900 includes outputting the dashboard for display when prompted. In an example, the prompt may include an explicit request to view the dashboard for the patient, entered by selection of an appropriate link/control button on the communication thread or selection of the patient's dashboard from a collaborative interface. For example, as shown in FIG. 2, a link to the dashboard may be displayed in the communication thread, and selecting the link may trigger display of the dashboard for that patient. In an example, the dashboard may be output for display automatically in response to a request from one or more of the VHAs. For example, the listening VHA may detect that a care provider is discussing the patient's current medical state and may automatically output the dashboard for display on the care provider's device so that the care provider may view patient medical information displayed in the dashboard that relates to the current medical state being discussed.

In some examples, the method 900 can also include outputting a patient score, the data quality index, a recommendation to obtain missing input data streams or missing features, or a combination thereof. For example, the data quality index can be output to any suitable display device associated with a patient, such as a care provider device, a hospital monitor, or the like. In some examples, an interface that includes the recommendation to obtain missing input data streams or missing features can be output to display devices associated with a patient. For example, an interface can display the data quality index and the recommendation for missing input data streams or features along with vital data, laboratory data, patient characteristics, or a combination thereof. In some examples, a first interface can display the data quality index and a link to a second interface that provides the recommendation for obtaining missing input data streams, missing features, or a combination thereof.

The process flow diagram of method 900 of FIG. 9 is not intended to indicate that all of the operations of blocks 902-912 of the method 900 are to be included in every example. Additionally, the process flow diagram of method 900 of FIG. 9 describes a possible order of executing operations. However, it is to be understood that the operations of the method 900 can be implemented in various orders or sequences. In addition, in some examples, the method 900 can also include fewer or additional operations. For example, the method 900 may include obtaining or otherwise retrieving data values for the missing input data stream or data values for missing parameters. For example, the one or more VHAs may be trained to process natural language inputs to determine the content of the input, e.g., determine if the input includes a request for information, and if the content incudes a request for information, the one or more VHAs may be trained to determine which patient the care provider is referring to and what information is being requested. In some examples, each VHA may process a received message to understand (in natural language) the intent of the message and determine if the intent of the message includes a task that the VHA is trained/configured to perform. In some examples, the server system may include a central entity configured to understand the intent of the message (e.g., from the natural language of the message) and determine which VHA is best configured to handle the request. Then, the mapping from intent (and VHAs) to a specific API of a specific VHA is one-to-one, e.g., only one VHA handles a specific intent (or intent-entity combination).

FIG. 10 is an example of a non-transitory machine-readable medium for detecting a position of a patient, in accordance with examples. The non-transitory, machine-readable medium 1000 can cause a processor 1002 to implement the functionalities of methods 700, 800, and 900. For example, a processor of a computing device (such as processor(s) 132 of server system 102 of FIG. 1), a care provider device 134, 136, or 138, or any other suitable device, can access the non-transitory, machine-readable media 1000.

In some examples, the non-transitory, machine-readable medium 1000 can include instructions to execute a data quality manager 1004. For example, the non-transitory, machine-readable medium 1000 can include instructions for the data quality manager 1004 that cause the processor 1002 to generate and provide a data quality index and a ranking of missing input data streams or missing features. The data quality manager 1004 can also implement the features of the data quality VHA 115 as described above in relation to FIG. 1. In some examples, the non-transitory, machine-readable medium 1000 can include instructions to implement any combination of the techniques of the method 700, 800, and 900 described above.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method for generating a data quality index comprising:
receiving a request to generate a patient score and an associated data quality index for a patient with a set of artificial intelligence instructions and one or more input data streams;
determining that at least one feature for the one or more input data streams is unavailable for the patient;
selecting one or more imputed values from a distribution for each of one or more features for the patient population;
calculating one or more initial scores using the one or more imputed values from the distribution as input for the at least one feature that is unavailable, the initial scores calculated using a set of artificial intelligence instructions, wherein the initial scores estimate a likelihood of an event or a clinical status;
calculating the data quality index based at least in part on the one or more initial scores, wherein the data quality index indicates a reliability of the patient score; and
providing the data quality index to an output device.

2. The method of claim 1, wherein the initial scores estimate a likelihood of a complication or an adverse risk for the patient.

3. The method of claim 1, wherein the one or more input data streams comprise at least one vital measurement from a vital data stream, at least one patient characteristic, at least one lab measurement value, or a combination thereof.

4. The method of claim 1, wherein the at least one feature comprises a minimum value, a maximum value, a standard deviation value, a median value, a mean value, or any combination thereof, wherein the at least one feature is determined based on the one or more data input streams within a predetermined time period.

5. The method of claim 1, wherein the set of artificial intelligence instructions implement a neural network, the neural network comprising one or more relative values or a feature importance for the at least one feature.

6. The method of claim 1, comprising determining a measurement threshold for the at least one feature, the measurement threshold representing a number of values used to calculate the at least one feature.

7. The method of claim 6, comprising identifying that the number of values for the at least one feature is below the minimum threshold.

8. The method of claim 7, comprising:
calculating a data availability score for the at least one feature;
calculating a data unavailability rank for the one or more input data streams with at least one missing feature, wherein the data unavailability rank is based at least in part on the data availability score and the relative value for each missing feature of the one or more data input streams; and
generating a recommendation output indicating a request for one or more unavailable input data streams or one or more unavailable measurements for an input data stream associated with one or more missing features based on the data unavailability rank.

9. The method of claim 8, comprising:
calculating an estimate of improvement in the data quality index following the addition of an unavailable input data stream.

10. The method of claim 8, comprising identifying one or more missing features with the data availability score above a first threshold value, the one or more missing features to be requested for an input data stream with the data unavailability rank above the first threshold value.

11. The method of claim 1, wherein providing the data quality index comprises transmitting the data quality index to an external device, displaying the data quality index, and generating an alert based on the data quality index, wherein the alert is provided by the external device.

12. The method of claim 1, wherein the data quality index is displayed with a patient score value indicating a likelihood that the patient is experiencing the complication or the adverse risk.

13. The method of claim 1, wherein calculating the data quality index further comprises:
calculating a variance value based on the one or more initial scores; and
determining the data quality index based at least in part on the variance value and a set of variance thresholds.

14. A system for generating a data quality index, comprising:
a processor to:
identify a distribution for each of one or more features for a patient population;
detect one or more input data streams for a patient from a medical device, wherein the one or more input data streams comprise at least one vital measurement from a vital data stream, at least one patient characteristic, at least one lab measurement value, or a combination thereof;
receive a request to generate a patient score and the data quality index for the patient with a set of artificial intelligence instructions and the one or more input data streams;
determine that at least one feature for the one or more input data streams is unavailable for the patient;
select one or more imputed values from the distribution for each of one or more features for the patient population;
calculate one or more initial scores using the one or more imputed values from the distribution as input for the at least one feature that is unavailable, the initial scores calculated using a set of artificial intelligence instructions, wherein the initial scores estimate a likelihood of an event or a clinical status;
calculate the data quality index based at least in part on the one or more initial scores, wherein the data quality index indicates a reliability of the patient score; and
provide the data quality index to an output device.

15. The system of claim 14, wherein the at least one feature comprises a minimum value, a maximum value, a standard deviation value, a median value, a mean value, or any combination thereof, wherein the at least one feature is determined based on the one or more data input streams within a predetermined time period.

16. The system of claim 14, wherein the set of artificial intelligence instructions implement a neural network, the neural network comprising one or more relative values for the at least one feature.

17. The system of claim 14, wherein the processor is to:
calculate a data availability score for the at least one feature;
calculate a data unavailability rank for the one or more input data streams with at least one missing feature, wherein the data unavailability rank is based at least in part on the data availability score and the relative value for each missing feature of the one or more data input streams; and
generate a recommendation output indicating a request for one or more unavailable input data streams or one or more unavailable measurements for an input data stream associated with one or more missing features based on the data unavailability rank.

18. The system of claim 17, wherein the processor is to identify one or more missing features with the data availability score above a first threshold value, the one or more missing features to be requested for an input data stream with the data unavailability rank above the first threshold value.

19. The system of claim 14, wherein the processor is to calculate the data quality index further by calculating a variance value based on the one or more initial scores and determining the data quality index based at least in part on the variance value and a set of variance thresholds.

20. A computer-readable medium for generating a data quality index, wherein the computer-readable medium comprises a plurality of instructions that, in response to execution by a processor, cause the processor to:
identify a distribution for each of one or more features for a patient population;
detect one or more input data streams for a patient from a medical device, wherein the one or more input data streams comprise at least one vital measurement from a vital data stream, at least one patient characteristic, at least one lab measurement value, or a combination thereof;
receive a request to generate a patient score and the data quality index for the patient with a set of artificial intelligence instructions and the one or more input data streams;
determine that at least one feature for the one or more input data streams is unavailable for the patient;
select one or more imputed values from the distribution for each of one or more features for the patient population;
calculate one or more initial scores using the one or more imputed values from the distribution as input for the at least one feature that is unavailable, the initial scores calculated using a set of artificial intelligence instructions, wherein the initial scores estimate a likelihood of an event or a clinical status;
calculate the data quality index based at least in part on the one or more initial scores, wherein the data quality index indicates an accuracy of the one or more initial scores, wherein calculating the data quality index comprises calculating a variance value based on the one or more initial scores and determining the data quality index based at least in part on a set of variance thresholds and the variance value; and
provide the data quality index to an output device.
